(19) United States
(12) Patent Application Publication
(10) Pub. No.: US 2024/0352022 A1
(43) Pub. Date: Oct. 24, 2024

(54) GROUP OF IMB-C5 SERIES COMPOUNDS HAVING ANTI-CORONAVIRUS ACTIVITY AND APPLICATION THEREOF

(71) Applicant: INSTITUTE OF MEDICINAL BIOTECHNOLOGY CHINESE ACADEMY OF MEDICAL SCIENCES, Beijing (CN)

(72) Inventors: Bin HONG, Beijing (CN); Xiaofang CHEN, Beijing (CN); Yu DU, Beijing (CN); Yuhuan LI, Beijing (CN); Xiaotian DING, Beijing (CN); Mengqian QIAO, Beijing (CN); Yihua LI, Beijing (CN); Kun WANG, Beijing (CN); Cong BIAN, Beijing (CN); Haiyan YAN, Beijing (CN); Shuo WU, Beijing (CN); Huiqiang WANG, Beijing (CN); Rongmei GAO, Beijing (CN); Li WANG, Beijing (CN); Lifei WANG, Beijing (CN); Hongmin SUN, Beijing (CN); Xingxing LI, Beijing (CN)

(21) Appl. No.: 18/706,087

(22) PCT Filed: Jun. 16, 2022
(86) PCT No.: PCT/CN2022/099223

§ 371 Date: Apr. 30, 2024

(30) Foreign Application Priority Data
Jun. 16, 2021 (CN) 202110668715.4

(51) Int. Cl. C07D 487/04; A61K 31/522; A61P 31/14; C07D 487/14

(52) U.S. Cl. CPC C07D 487/04; A61K 31/522; A61P 31/14; C07D 487/14

(57) ABSTRACT

The present invention relates to a group of IMB-C5 series compounds having anti-coronavirus activity and the application thereof. The structure of the IMB-C5 series compounds is as shown in formula (1). The application is an application of the IMB-C5 series compounds in preparing a drug. The drug is a drug for inhibiting coronavirus, and is preferably a drug for treating human coronavirus infection. The coronavirus is preferably human α-coronavirus or human β-coronavirus, especially novel coronavirus (SARS-COV-2).

### TECHNICAL FIELD

The present disclosure belongs to the field of pharmaceutical biotechnology, and specifically, relates to a group of IMB-C5 series compounds having anti-coronavirus activity and application thereof.

### BACKGROUND

Acute infectious diseases pose a major threat to public health, among which acute respiratory infection is a main cause of morbidity and mortality caused by infectious disease worldwide. Human coronavirus is one of the common pathogens that cause acute respiratory infection, and can cause zoonosis and may lead to a global pandemic. Since the 21^{st} century, the world has experienced three epidemic outbreaks caused by highly pathogenic coronaviruses, including severe acute respiratory syndrome (SARS) in 2002, Middle East respiratory syndrome (MERS) in 2012 and COVID-19 caused by severe acute respiratory syndrome coronavirus 2 (SARS-COV-2) in late 2019. COVID-19 has become the fifth global pandemic recorded since the influenza pandemic in 1918 and the first coronavirus pandemic in human history, causing unprecedented damage to global health and economic development^{[1]}. The “two-pronged approach” of vaccination and drug treatment will become the main means of fighting the virus at the next stage. Small molecule drugs have become the focus of antiviral drug research and development because of their advantages such as clear targets, easy large-scale production and distribution, and convenient oral administration.

Coronavirus is a class of enveloped positive-sense single-stranded RNA virus. Currently, there are 7 known coronaviruses that can infect human, including HCoV-229E and HCoV-NL63 of the genus α-coronavirus, and HCoV-OC43, HCoV-HKU1, SARS-COV, MERS-COV and SARS-COV-2 of the genus β-coronavirus, among which SARS-COV, MERS-COV and SARS-COV-2 are highly pathogenic. The coronavirus genome is about 26-32 Kb and encodes 4 structural proteins, including spike protein (S protein), envelope protein (E protein), membrane protein (M protein) and nucleocapsid protein (N protein), and non-structural proteins, such as RNA-dependent RNA polymerase (RdRp), 3-chymotrypsin like protease (3C-like protease, 3CLpro, also known as main protease (Mpro)), papain-like protease (PLpro) and the like. Two small molecule oral drugs, Paxlovid and Molnupiravir (MNP), targeting 3CLpro and RdRp, respectively, have recently been approved for marketing, and can decrease hospitalization rate and mortality of mild/non-severe patients at a high risk of hospitalization to some extent. These drugs that directly target the viral targets have high specificity, but they also face the challenges of rapid virus mutation and development of drug resistance.

Virus needs to invade host cells for survival and proliferation. Therefore, the strategy of developing an antiviral drug against a host target has increasingly received attention from researchers, which makes it possible to develop novel antiviral drugs which have broad-spectrum antiviral effects and are less likely to develop drug resistance. Virus replicates itself by hijacking and utilizing particular host proteins, and its energy supply and substrate metabolism, etc. also depend on the host. Lipid is a fundamental cellular component that plays an important biological role in composing cell structure, serving as a signaling molecule and energy storage, etc. and is crucial in the viral life cycle^{[2]}. SARS-COV-2 is a virus enveloped by a lipid bilayer. Lipid is involved in the processes of the viral life cycle, including fusion of the viral membrane with the host cell, viral replication, viral endocytosis and exocytosis, etc. Coronavirus replication can be interfered by targeting lipid metabolism in the host cells, which is a new idea for developing novel anti-coronavirus drugs. It is found in the clinical data study that the levels of certain lipids in the plasma of COVID-19 patients are related to the severity of infection^{[3]}. Some experts have proposed that lipid-lowering therapy such as statins can be one of the therapeutic strategies for complications in severe COVID-19 patients^{[4]}.

IMB-C5 is a novel small molecule inhibitor for proprotein convertase subtilisin/kexin type 9 (PCSK9), which was obtained by utilizing a PCSK9 transcriptional inhibitor screening model in our laboratory. IMB-C5 has been validated to have a good effect on lowering blood lipids and inhibiting atherosclerotic plaque formation^{[5]}. Through a series of anti-coronavirus studies, it was found that IMB-C5 has a good inhibitory effect on the replication of common coronaviruses (HCoV-229E and HCoV-OC43), and also has an inhibitory effect on SARS-COV-2, suggesting that its antiviral mechanism may be related to the regulation of lipid metabolism. Based on the structure of IMB-C5 compound, we have performed a series of modifications in the chemical structure and obtained some active compounds with a better antiviral activity and a higher therapeutic index, which are expected to be developed as novel anti-coronavirus drugs. Such compounds, as host-targeting drugs, can also be combined with inhibitors that target virus such as 3CLpro and RdRp to form an antiviral “cocktail” to further improve the treatment for COVID-19.

### REFERENCES

[1] Liu YC, Kuo RL, Shih SR. COVID-19: The first documented coronavirus pandemic in history. Biomed J, 2020, 43(4):328-333.
[2] Abu-Farha M, Thanaraj TA. Qaddoumi MG, Hashem A, Abubaker J, Al-Mulla F. The role of lipid metabolism in COVID-19 virus infection and as a drug target. Int J Mol Sci, 2020, 21(10):3544.
[3] Hu X, Chen D, Wu L, He G Ye W. Declined serum high density lipoprotein cholesterol is associated with the severity of COVID-19 infection. Clin Chun Acta, 2020, 510:105-110.
[4] Morawietz H, Julius U, Bornstein SR. Cardiovascular diseases, hpid-lowering therapies and European registries in the COVID-19 pandemic. Cardiovasc Res. 2020, 116(10):e122-e125.
[5] Wang X, Chen X, Zhang X, et al. A small-molecule inhibitor of PCSK9 transcription ameliorates atherosclerosis through the modulation of FoxO1/3 and HNF1α. EBioMedicine. 2020, 52:102650.

### SUMMARY

Firstly, the present disclosure relates to a compound of Formula (1),

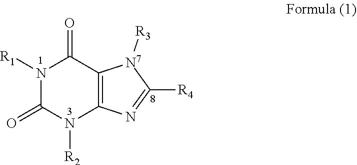

wherein,
R₁ is alkyl; substituted alkyl; or a substituent comprising alkenyl, alkynyl, epoxy alkyl, benzyl, pyridine and the like; preferably, R₁ is C1-C5 alkyl; C1-C5 monoalkenyl; C1-C5 monoalkynyl; alkyl comprising pyridine ring(s), benzene ring(s) or substituted benzene ring(s); or Boc-NH-linked C1-C3 alkyl;
R₂ is alkyl; substituted alkyl; or a substituent comprising alkenyl, alkynyl or benzyl; preferably, R₂ is methyl or benzyl;
R₃ is alkyl; substituted alkyl; epoxy alkyl; benzyl; substituted benzyl; thenyl; substituted phenethyl and the like; preferably, R₃ is epoxy hexane methyl; formate benzyl; methoxyl; fluoro-methoxyl or fluoro-methyl modified benzyl; monohalogenated or polyhalogenated benzyl; C1-C5 alkyl substituted benzyl; nitro-substituted benzyl; thenyl; or substituted phenylethyl;
R₄ is —NH—R₅; benzyl or substituted benzyl; imidazolyl; or substituted piperazinyl;
R₅ is alkyl; substituted alkyl; hydroxyalkyl; amide-containing alkyl; ester-containing alkyl, alkoxy, and the like; preferably, R₅ is straight or branched C1-C5 hydroxyalkyl, alkoxy alkyl; halogen atom and/or methyl or fluoro-methyl substituted benzene ring and/or benzyl; amide-containing alkyl; or ester-containing alkyl.

More preferably, the compound is IMB-ZH-2, IMB-ZH-11, IMB-ZH-12, IMB-ZHC-2, IMB-ZHC-15, IMB-2-26, IMB-2-31, IMB-2-32, IMB-3-19, IMB-4-6, IMB-4-12, IMB-4-13, IMB-ZHB-4x, IMB-2-3, IMB-2-8, IMB-68, IMB-82, IMB-83, IMB-84, IMB-85, IMB-92, IMB-93, IMB-3-6, IMB-3-13, IMB-3-15, IMB-3-16, IMB-3-30, IMB-3-45, IMB-3-46, IMB-3-47, IMB-3-57, IMB-3-58, IMB-3-71, IMB-3-72 or IMB-3-81.

The present disclosure further relates to use of the compound of Formula (1) in the preparation of a medicament for inhibiting coronavirus

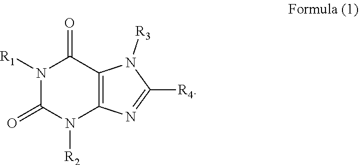

Preferably, the coronavirus is a human coronavirus; more preferably, the coronavirus is a human α-coronavirus or a human β-coronavirus; most preferably, the coronavirus is HCoV-229E, HCoV-OC43 or SARS-COV-2.

The present disclosure further relates to use of the compound of Formula (1) in the preparation of a combined medicament for inhibiting coronavirus, wherein the combined medicament further comprises an agent that targets a further viral target; preferably, the further viral target is 3CLpro and/or RdRp and the like

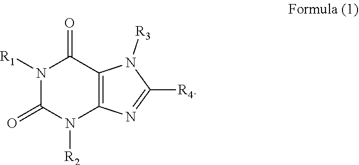

Preferably, the coronavirus is a human coronavirus; more preferably, the coronavirus is a human α-coronavirus or a human β-coronavirus; most preferably, the coronavirus is HCoV-229E, HCoV-OC43 or SARS-COV-2.

The present disclosure further relates to a pharmaceutical composition and/or formulation for treating coronavirus, comprising a therapeutically effective amount of the compound of Formula (1), and an optional pharmaceutical excipient/diluent.

The present disclosure further relates to a method for treating a disease caused by coronavirus infection, comprising administering to a patient a therapeutically effective amount of the compound of Formula (1) or the pharmaceutical composition and/or formulation comprising the compound of Formula (1).

The present disclosure further relates to a method for synthesizing the compound of Formula (1), specifically comprising the following steps:

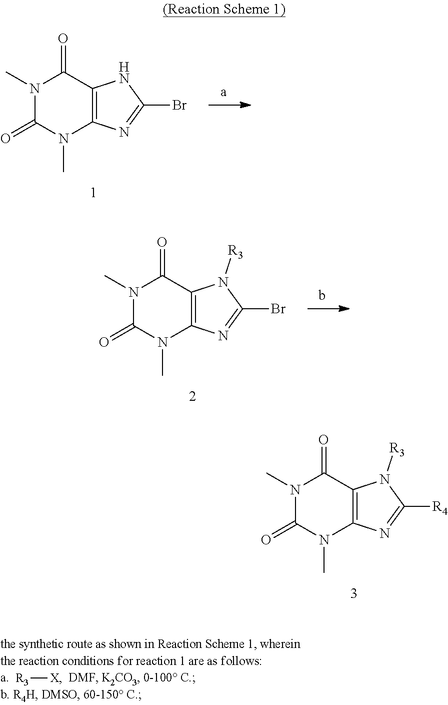

or the synthetic route as shown in Reaction Scheme 2:

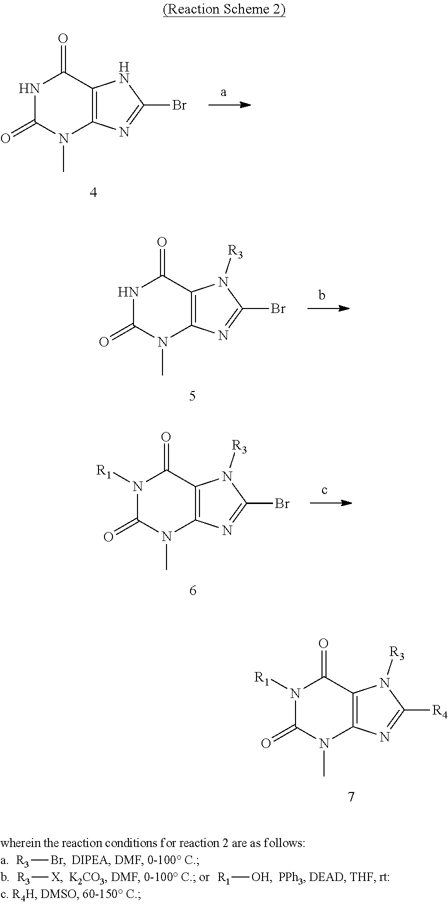

or the synthetic route as shown in Reaction Scheme 3:

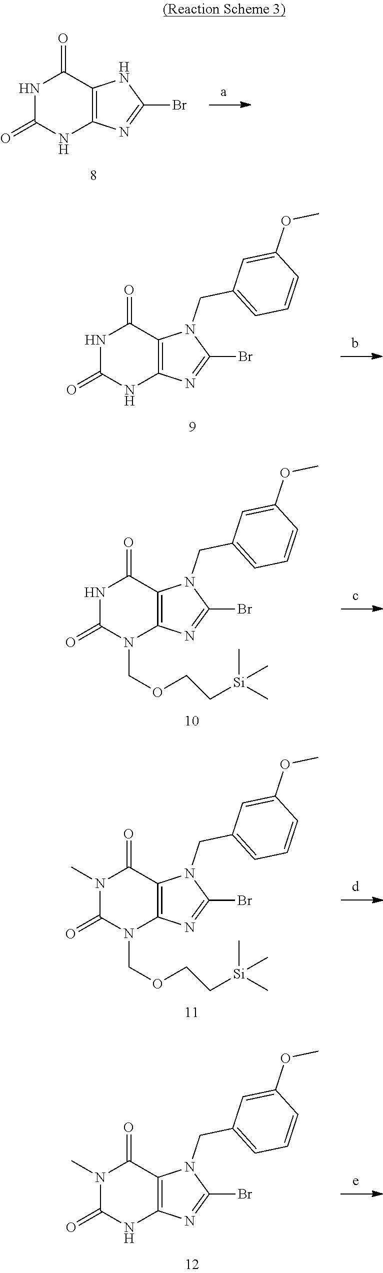 [SMILES: O=c1[nH]c(=O)c2[nH]c(Br)nc2[nH]1 ; COc1cccc(Cn2c(Br)nc3[nH]c(=O)[nH]c(=O)c32)c1 ; COc1cccc(Cn2c(Br)nc3c2c(=O)[nH]c(=O)n3COCC[Si](C)(C)C)c1 ; COc1cccc(Cn2c(Br)nc3c2c(=O)n(C)c(=O)n3COCC[Si](C)(C)C)c1 ; COc1cccc(Cn2c(Br)nc3[nH]c(=O)n(C)c(=O)c32)c1]

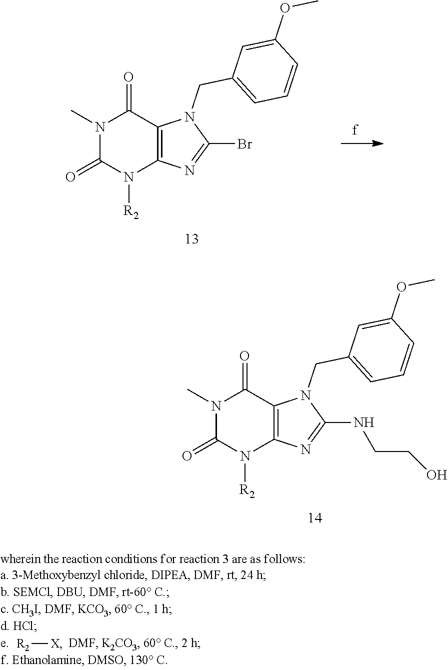

### BRIEF DESCRIPTION OF THE DRAWINGS

FIG. 1 shows the effect of IMB-C5 on mRNA level of coronavirus HCOV-229E N protein in Huh7 and Huh7.5 cells.

Con represents Control, which indicates the no-treatment virus control; RBV represents Ribavirin.

FIG. 2 shows the effect of IMB-C5 on dsRNA production during replication of HCoV-229E in Huh7 cells (immunofluorescence assay).

Mock represents the blank control of untreated cells; Con represents Control, which indicates the no-treatment virus control; RBV represents Ribavirin; RDV represents Remdesivir.

FIG. 3 shows the effect of IMB-C5 on mRNA level of coronavirus HCoV-OC43 N protein in C3A cells.

Con represents Control, which indicates the no-treatment virus control; RBV represents Ribavirin.

FIG. 4 shows the effect of IMB-C5 on N protein level of coronavirus HCoV-OC43 in C3A cells (Western Blotting).

Mock represents the blank control of untreated cells; Con represents Control, which indicates the no-treatment virus control; RBV represents Ribavirin.

FIG. 5 shows the effect of IMB-C5 series compounds on N protein level of human coronavirus HCoV-229E in Huh7 cells.

Mock represents the blank control of untreated cells; Con represents Control, which indicates the no-treatment virus control; MNP represents Molnupiravir. In the figure, Compound IMB-2-5 is marked as 2-5, and the rest are marked in the same way.

FIG. 6 shows the toxicity assay results of IMB-C5 and IMB-85 in different cells.

FIG. 7 shows the effect of IMB-C5 and IMB-85 on mRNA level of human coronavirus HCoV-229E N protein in Huh7 cells.

Con represents Control, which indicates the no-treatment virus control; RBV represents Ribavirin.

FIG. 8 shows the effect of IMB-C5 and IMB-85 on N protein level of human coronavirus HCoV-229E in Huh7 cells (Western Blotting).

Mock represents the blank control of untreated cells; Con represents Control, which indicates the no-treatment virus control; RBV represents Ribavirin.

FIG. 9 shows the effect of IMB-C5 and IMB-85 on dsRNA production during replication of HCoV-229E in Huh7 cells (immunofluorescence assay).

Mock represents the blank control of untreated cells; Con represents Control, which indicates the no-treatment virus control; RBV represents Ribavirin.

FIG. 10 shows the effect of IMB-C5 and IMB-85 on N protein level of coronavirus HCoV-229E in Huh7 cells (A) and Huh7.5 cells (B) (Western Blotting).

Mock represents the blank control of untreated cells; Con represents Control, which indicates the no-treatment virus control; MNP represents Molnupiravir.

FIG. 11 shows the effect of IMB-C5 and IMB-85 on N protein level of coronavirus HCoV-OC43 in C3A cells (Western Blotting).

Mock represents the blank control of untreated cells; Con represents Control, which indicates the no-treatment virus control; MNP represents Molnupiravir.

FIG. 12 shows the results of the time-of-addition experiments of IMB-C5 on coronavirus (the dsRNA level of the virus determined by immunofluorescence assay).

Con represents Control, which indicates the no-treatment virus control; Mock represents the blank control of untreated cells.

FIG. 13 shows the results of the time-of-addition experiments of IMB-85 on coronavirus (the N protein level of the virus determined by Western Blotting).

Mock represents the blank control of untreated cells; Con represents Control, which indicates the no-treatment virus control.

FIG. 14 shows the results of the time-of-addition experiments of IMB-85 on coronavirus (the dsRNA level of the virus determined by immunofluorescence assay).

Mock represents the blank control of untreated cells; Con represents Control, which indicates the no-treatment virus control.

### DETAILED DESCRIPTION

### Experimental Materials

### 1. Cell Strains and Viruses

Human hepatocellular carcinoma cells C3A (ATCC, CRL-10741), preserved in our laboratory.

Human hepatocellular carcinoma cells Huh7, preserved in our laboratory.

Human hepatocellular carcinoma cells Huh7.5, preserved in our laboratory.

African green monkey kidney cells Vero E6 (ATCC, CRL-1586), preserved in our laboratory.

Human coronavirus HCoV-229E (ATCC, VR-740), preserved in our laboratory.

Human coronavirus HCoV-OC43 (ATCC, VR-1558), preserved in our laboratory.

Human coronavirus SARS-COV-2 Beta variant, preserved in Guangdong Provincial Center for Disease Control.

### 2. Compounds

Ribavirin (RBV) injection, as a positive control, was purchased from Tianjin KingYork Group Hubei Tianyao Pharmaceutical Co. LTD (batch number: 31712252, specification: 100 mg/mL). Remdesivir (RDV), as a positive control, was purchased from MedChemExpress LLC (Cat. No. HY-104077). Molnupiravir (MNP), as a positive control, was purchased from Shanghai Taoshu Biotechnology Co., Ltd (Cat. No. T8309).

### 3. Primers for Reverse Transcription Quantitative PCR (RT-qPCR)

F=Forward primer, R=Reverse primer, P=Probe, NP=Nucleocapsid protein

| Name | Sequence (5′→>3′) |
|---|---|
| GAPDH-F | CTCTGGAAAGCTGTGGCGTGATG |
| GAPDH-R | ATGCCAGTGAGCTTCCCGTTCAG |
| HCoV-229E-NP-F | GACCRATCCTGTCACCTCTGAC |
| HCoV-229E-NP-R | GGGCATTYTGGACAAAKCGTCTACG |
| GAPDH-2-F | CGGAGTCAACGGATTTGGTCGTAT |
| GAPDH-2-R | AGCCTTCTCCATGGTGGTGAAGAC |
| GAPDH-2-P | TAMRA-CCGTCAAGGCTGAGAACGG-BHQ2 |
| HCoV-OC43-NP-F | CGATGAGGCTATTCCGACTAGGT |
| HCoV-OC43-NP-R | CCTTCCTGAGCCTTCAATATAGTAACC |
| HCoV-OC43-NP-P | TAMRA-TCCGCCTOGCACGGTACTCCCT-BHQ2 |

### 4. Antibodies

Antibody against coronavirus HCoV-OC43 nucleocapsid protein (NP) (mouse monoclonal antibody, MAB9013, Millipore).

Antibody against HCoV-229E NP (rabbit polyclonal antibody, 40640-T62, Sino Biological).

Antibody against human glyceraldehyde-3-phosphate dehydrogenase (GAPDH) (mouse monoclonal antibody, ZB2305, ZSGB-BIO).

Antibody against double strand RNA (dsRNA) (mouse monoclonal antibody J2, SCICONS).

Fluorescein Isothiocyanate (FITC)-labeled goat anti-mouse IgG (H+L) (HS211, TransGen).

### 5. Reagents

CCK-8 Cell Counting Kit (CCK-8 Kit), purchased from Nanjing Vazyme Biotech Co., Ltd (Cat. No. A311).

### EXAMPLE 1. SYNTHESIS METHODS AND NMR DATA OF TARGET COMPOUNDS

### 1.1. Reaction Route for N3 Modification and Synthesis of Intermediate 12

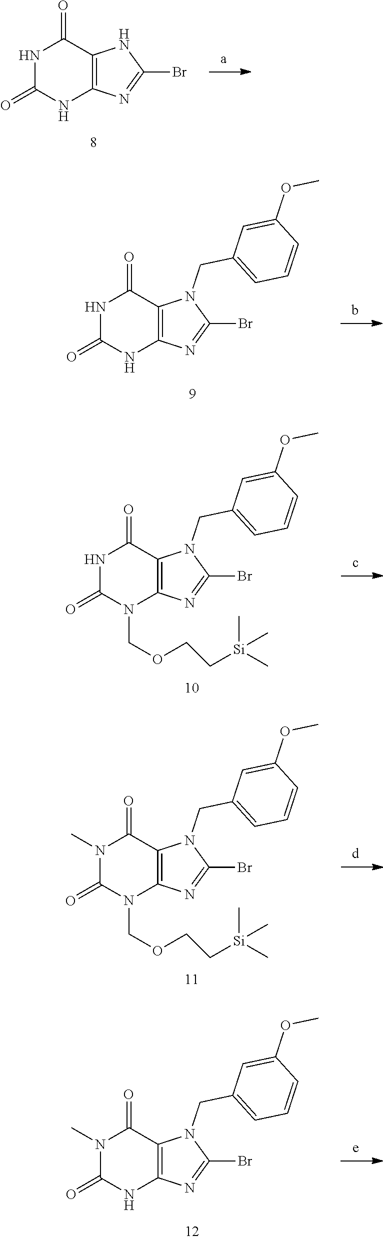 [SMILES: O=c1[nH]c(=O)c2[nH]c(Br)nc2[nH]1 ; COc1cccc(Cn2c(Br)nc3[nH]c(=O)[nH]c(=O)c32)c1 ; COc1cccc(Cn2c(Br)nc3c2c(=O)[nH]c(=O)n3COCC[Si](C)(C)C)c1 ; COc1cccc(Cn2c(Br)nc3c2c(=O)n(C)c(=O)n3COCC[Si](C)(C)C)c1 ; COc1cccc(Cn2c(Br)nc3[nH]c(=O)n(C)c(=O)c32)c1]

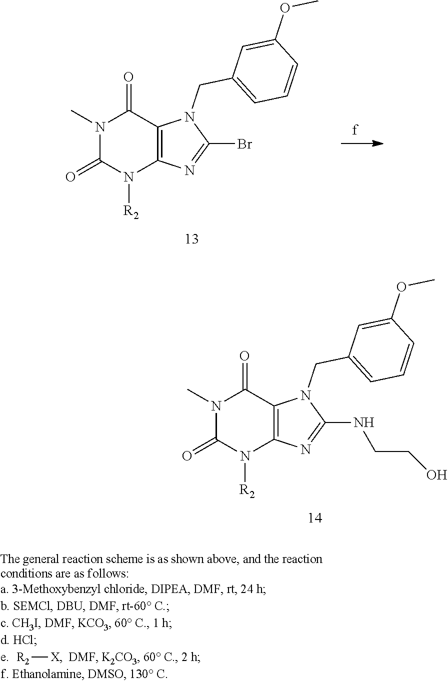

Specific steps are described as follows:

### Step 1:

Under the protection of nitrogen, 8-bromoxanthine (compound 8 in the reaction scheme, 1.0 equiv) was added into a round-bottom flask and dissolved in dry DMF (3.0 mL), followed by addition of anhydrous K₂CO₃ or NaH (1.2 equiv). The obtained solution was added with methoxybenzyl chloride (1.0 equiv), sealed and reacted at 0° C. for about 24 h until the reaction was complete and raw material spots disappear (monitored by TLC). The reaction solution was extracted with EA, washed with water, washed with saturated NaCl solution, dried and concentrated over anhydrous Na₂SO₄, and purified by column chromatography to obtain a product (compound 9 in the reaction scheme) (PE/EA=3/1−1/1).

### Step 2:

Under the protection of nitrogen, the product obtained in step 1 (compound 9 in the reaction scheme, 1.0 equiv) was added into a round-bottom flask and dissolved in dry DMF (3.0 mL), followed by addition of DBU (1.2 equiv). The obtained solution was added with 2-(trimethylsilyl) ethoxymethyl chloride (1.0 equiv), sealed and reacted at 0° C. for about 24 h until the reaction was complete and raw material spots disappear (monitored by TLC). The reaction solution was extracted with EA, washed with water, washed with saturated NaCl solution, dried and concentrated over anhydrous Na₂SO₄ to obtain a crude product (compound 10 in the reaction scheme).

### Step 3:

Under the protection of nitrogen, the crude product obtained in step 2 (compound 10 in the reaction scheme, 1.0 equiv) was added into a round-bottom flask and dissolved in dry DMF (3.0 mL), followed by addition of anhydrous K₂CO₃ or NaH (1.2 equiv). The obtained solution was added with methyl iodide (1.0 equiv), sealed and reacted at 60° C. for about 2 h until the reaction was complete and raw material spots disappear (monitored by TLC). The reaction solution was extracted with EA, washed with water, washed with saturated NaCl solution, dried and concentrated over anhydrous Na₂SO₄ to obtain a crude product (compound 11 in the reaction scheme).

### Step 4:

Under the protection of nitrogen, the crude product obtained in step 3 (compound 11 in the reaction scheme, 1.0 equiv) was added into a round-bottom flask and dissolved in dry DCM (3.0 mL). The obtained solution was added with TFA (1.0 mL) and stirred at room temperature for about 1 h until the reaction was complete and raw material spots disappear (monitored by TLC). The reaction solution was adjusted to a neutral pH with 0.5 N NaOH, extracted with EA, washed with water, washed with saturated NaCl solution, dried and concentrated over anhydrous Na₂SO₄, and purified by column chromatography to obtain a product (compound 12 in the reaction scheme) (PE/EA=3/1−1/1).

### 1.2. General Synthesis Methods

### General Synthesis Method 1:

Specifically, under the protection of nitrogen, 8-bromotheophylline (compound 1, 1.0 equiv) was added into a round-bottom flask and dissolved in dry DMF (3.0 mL), followed by addition of anhydrous K₂CO₃ or NaH (1.2 equiv). The obtained solution was added with halide (1.2 equiv), sealed and reacted at 60° C. for about 2 h until the reaction was complete and raw material spots disappear (monitored by TLC). The reaction solution was extracted with EA, washed with water, washed with saturated NaCl solution, dried and concentrated over anhydrous Na₂SO₄, and purified by column chromatography to obtain a product (compound 2) (PE/EA=3/1−1/1).

### General Synthesis Method 2 (N1 Modification):

Specifically, under the protection of nitrogen, 8-bromo-3-methyl-3,7-dihydro-purine-2,6-dione (compound 4, 1.0 equiv) was added into a round-bottom flask and dissolved in dry DMF (3.0 mL), followed by addition of anhydrous K₂CO₃ or NaH (1.2 equiv). The obtained solution was added with methoxybenzyl bromide (1.0 equiv), sealed and reacted at 60° C. for about 2 h until the reaction was complete and raw material spots disappear (monitored by TLC). The reaction solution was extracted with EA, washed with water, washed with saturated NaCl solution, dried and concentrated over anhydrous Na₂SO₄, and purified by column chromatography to obtain a product (compound 5) (PE/EA=3/1−1/1).

### General Synthesis Method 3:

Under the protection of nitrogen, compound 5 obtained in general synthesis method 2 or intermediate 12 (1.0 equiv) was added into a round-bottom flask and dissolved in dry DMF (3.0 mL), followed by addition of anhydrous K₂CO₃ or NaH (1.2 equiv). The obtained solution was added with different halide (3.0 equiv), sealed and reacted at 70° C. for about 7 h until the reaction was complete and raw material spots disappear (monitored by TLC). The reaction solution was extracted with EA, washed with water, washed with saturated NaCl solution, dried and concentrated over anhydrous Na₂SO₄, and purified by column chromatography to obtain a product (compound 6 or compound 13) (PE/EA=3/1−1/1).

### General Synthesis Method 4:

Under the protection of nitrogen, compound 2, 6 or 13 (1.0 equiv) obtained in general synthesis method 1 or 3 was added into a round-bottom flask and dissolved in dry DMF (3.0 mL), followed by addition of anhydrous K₂CO₃ or NaH (1.2 equiv). The obtained solution was added with an amine compound (3.0 equiv), sealed and reacted at 130° C. for about 13 h until the reaction was complete and raw material spots disappear (monitored by TLC). The reaction solution was extracted with EA, washed with water, washed with saturated NaCl solution, dried and concentrated over anhydrous Na₂SO₄, and purified by column chromatography to obtain a product (compound 3, 7 or 14) (DCM/MeOH=30/1−20/1).

### 1.3. Synthetic Routes

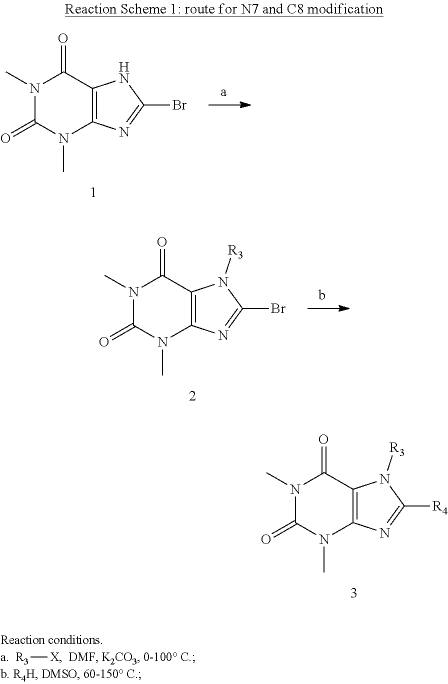

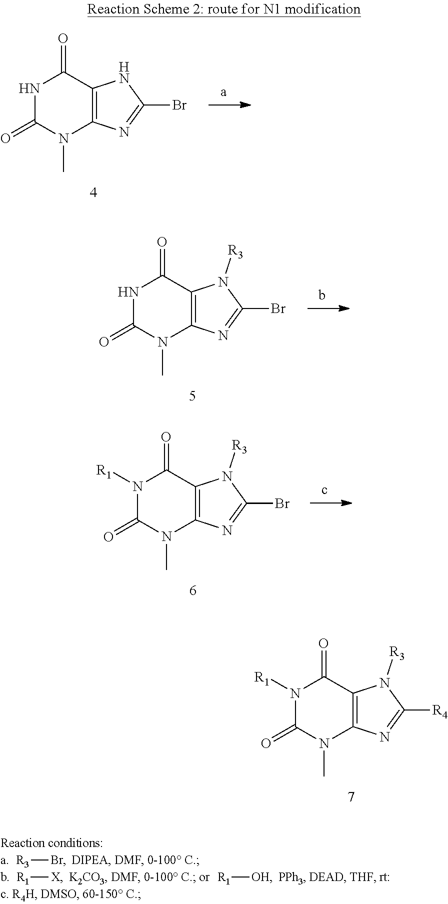

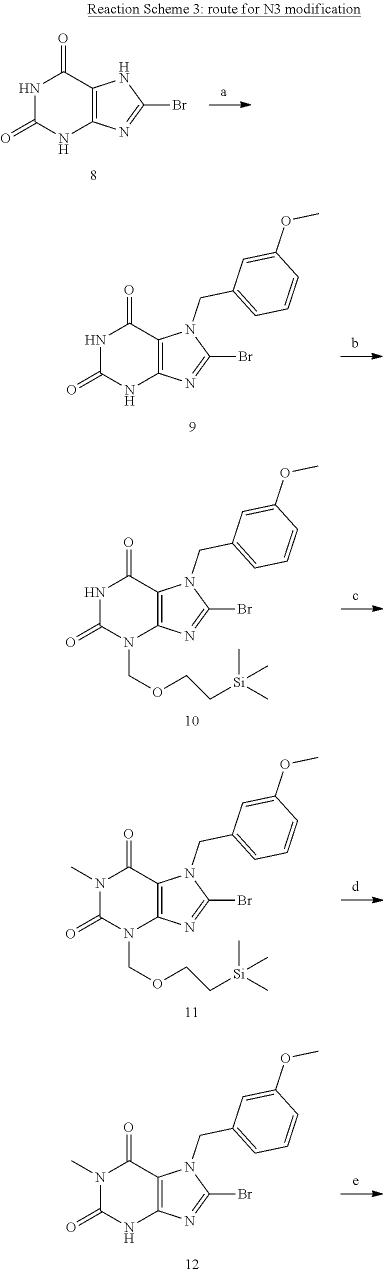 [SMILES: O=c1[nH]c(=O)c2[nH]c(Br)nc2[nH]1 ; COc1cccc(Cn2c(Br)nc3[nH]c(=O)[nH]c(=O)c32)c1 ; COc1cccc(Cn2c(Br)nc3c2c(=O)[nH]c(=O)n3COCC[Si](C)(C)C)c1 ; COc1cccc(Cn2c(Br)nc3c2c(=O)n(C)c(=O)n3COCC[Si](C)(C)C)c1 ; COc1cccc(Cn2c(Br)nc3[nH]c(=O)n(C)c(=O)c32)c1]

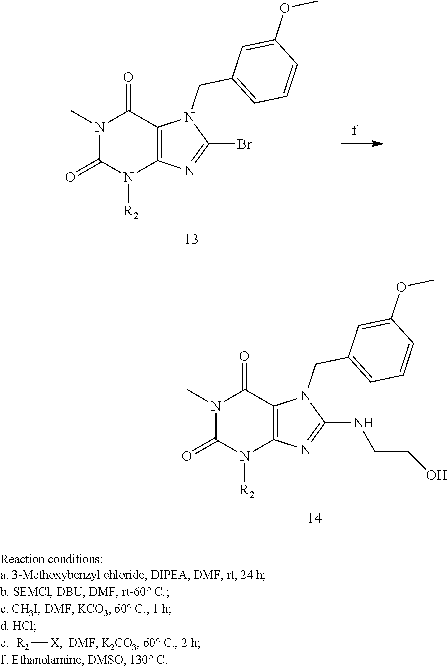

### 1.4. Structures and NMR Data of the Synthesized Compounds

### C8-Modified Compounds are as Follows:

### IMB-ZH-2

(S)-8-((1-hydroxypropan-2-yl)amino)-7-(3-methoxybenzyl)-1,3-dimethyl-3,7-dihydro-1H-purine-2,6-dione

### Appearance: White Solid; Yield: 75%.

¹H NMR (500 MHz, DMSO-d₆) δ=7.22 (t, J=7.8, 1H), 6.88 (d, J=7.8, 1H), 6.82 (s, 2H), 6.77 (d, J=7.3, 1H), 5.28 (q, J=16.0, 2H), 4.74 (s, 1H), 3.98-3.92 (m, 1H), 3.71 (s, 3H), 3.47 (d, J=4.9, 1H), 3.35 (s, 3H), 3.15 (s, 3H), 1.14 (d, J=6.3, 3H), ¹³C NMR (126 MHz, DMSO-d₆) δ=159.38, 153.67, 152.81, 151.10, 149.00, 138.85, 129.74, 119.23, 113.04, 112.68, 101.15, 64.49, 55.07, 50.64, 45.26, 29.45, 27.32, 17.64. HRMS (ESI) Calcd for C₁₈H₂₄N₅O₄ [M+H]⁺ 374.1828; Found 374.1822.

### IMB-ZH-6

8-((3-hydroxypropyl)amino)-7(3-methoxybenzyl)-1,3-dimethyl-3,7-dihydro-1H-purine-2,6-dione

### Appearance: White Solid; Yield: 86%.

¹H NMR (500 MHz, DMSO-d₆) δ=7.23 (t, J=8.1, 1H), 7.14 (s, 1H), 6.82 (s, 2H), 6.76 (d, J=7.3, 1H), 5.26 (s, 2H), 4.48 (s, 1H), 3.71 (s, 3H), 3.44 (d, J=5.1, 2H), 3.39 (d, J=5.8, 2H), 3.36 (s, 3H), 3.34 (s, 1H), 3.16 (s, 3H), 1.77-1.61 (m, 2H). ¹³C NMR (126 MHz, DMSO-d₆) δ=159.41, 153.97, 152.87, 151.08, 148.93, 138.70, 129.78, 119.16, 113.15, 112.58, 101.29, 58.31, 55.09, 45.23, 40.51, 32.52, 29.44, 27.33. HRMS (ESI) Calcd for C₁₈H₂₄N₅O₄ [M+H]⁺ 374.1828; Found 374.1824.

### IMB-ZH-10

7-(3-methoxybenzyl)-8-((2-methoxyethyl)amino)-1,3-dimethyl-3,7-dihydro-1H-purine-2,6-dione

### Appearance: White Solid; Yield: 73%.

¹H NMR (500 MHz, DMSO-d₆) δ=7.28 (d, J=5.0, 1H), 7.22 (t, J=7.8, 1H), 6.82 (d, J=7.4, 2H), 6.77 (d, J=7.5, 1H), 5.26 (s, 2H), 3.71 (s, 3H), 3.48 (dd, J=9.8, 4.5, 4H), 3.35 (s, 3H), 3.24 (s, 3H), 3.16 (s, 3H). ¹³C NMR (126 MHz, DMSO-d₆) δ=159.29, 153.85, 152.76, 150.99, 148.87, 138.59, 129.67, 119.02, 113.17, 112.38, 101.19, 62.02, 55.52, 54.97, 45.10, 42.05, 29.33, 27.22. HRMS (ESD) Calcd for C₁₈H₂₄N₅O₄ [M+H]⁺ 374.1828; Found 374.2.

### IMB-ZH-11

8-((4-fluoro-3-(trifluoromethyl)benzyl)amino)-7-(3-methoxybenzyl)-1,3-dimethyl-3,7-dihydro-1H-purine-2,6-dione

### Appearance: White Solid; Yield: 73%.

¹H NMR (500 MHz, DMSO-d₆) δ=7.83 (t, J=5.5, 1H), 7.70 (d, J=6.7, 1H), 7.65 (s, 1H), 7.43 (t, J=9.6, 1H), 7.21 (t, J=7.8, 1H), 6.83 (d, J=8.2, 1H), 6.79 (s, 1H), 6.74 (d, J=7.4, 1H), 5.29 (s, 2H), 4.58 (d, J=5.4, 2H), 3.68 (s, 3H), 3.33 (d, J=7.8, 4H), 3.16 (s, 3H). ¹³C NMR (151 MHz, DMSO-d₆) δ=159.33, 153.30, 152.93, 150.91, 148.45, 138.29, 136.69, 134.22, 129.61, 126.12, 123.52, 118.90, 117.06, 116.91, 116.21, 113.06, 112.42, 101.59, 54.89, 45.27, 44.61, 29.23, 27.19. HRMS (ESI) Calcd for C₂₃H₂₂N₅O₃F₄ [M+H]⁺ 270.1658; Found 492.1645.

### IMB-ZH-12

(R)-8-((1-hydroxy-3-methylbutan-2-yl)amino)-7-(3-methoxybenzyl)-1,3-dimethyl-3,7-dihydro-1H-purine-2,6-dione

### Appearance: White Solid; Yield: 82%.

¹H NMR (500 MHz, DMSO-d₆) δ=7.21 (t, J=7.8, 1H), 6.85 (s, 1H), 6.80 (t, J=8.6, 2H), 6.70 (d, J=8.6, 1H), 5.41 (d, J=16.0, 1H), 5.28 (d, J=15.9, 1H), 4.59 (s, 1H), 3.74 (s, 1H), 3.70 (s, 3H), 3.51 (d, J=5.9, 2H), 3.33 (d, J=13.5, SH), 3.16 (s, 3H), 1.91 (dd, J=13.3, 6.7, 1H), 0.80 (dd, J=14.1, 6.7, 6H). ¹³C NMR (151 MHz, DMSO-d₆) δ=159.28, 154.28, 152.67, 350.99, 148.78, 138.71, 129.52, 119.25, 112.59, 112.59, 101.02, 61.04, 60.05, 54.94, 45.08, 29.26, 28.49, 27.15, 19.43, 18.28. HRMS (ESI) Calcd for C₂₀H₂₈N₅O₄ [M+H]⁺ 402.2141; Found 402.2137.

### IMB-ZHC-2

methyl(7-(3-methoxybenzyl)-1,3-dimethyl-2,6-dioxo-2,3,6,7-tetrahydro-1H-purin-8-yl)glycinate

### Appearance: Brown Solid; Yield: 76%.

¹H NMR (500 MHz, CDCl₃) δ=6.97-6.88 (m, 2H), 6.84 (d, J=8.1, 1H), 5.52 (d, J=5.2, 2H), 3.79 (s, 3H), 3.60-3.53 (m, 3H), 3.39 (t, J=8.1, 3H), 1.54 (s, 2H). ¹³C NMR (151 MHz, CDCl₃) δ=160.07, 154.41, 151.41, 148.42, 147.47, 136.53, 130.13, 127.98, 320.08, 113.91, 113.70, 109.07, 55.40, 50.26, 49.36, 49.26, 30.01, 28.24. HRMS (ESI) Calcd for C₁₈H₂₂N₅O₅ [M+H]⁺ 388.1615; Found 388.1632.

### IMB-ZHC-6

(R)-8-((2-hydroxypropyl)amino)-7-(3-methoxybenzyl)-1,3-dimethyl-3,7-dihydro-1H-purine-2,6-dione

### Appearance: White Solid; Yield: 76%.

¹H NMR (600 MHz, CDCl₃) δ=6.82 (dd, J=11.3, 5.0, 2H), 6.78 (s, 1H), 5.32 (s, 2H), 4.71 (s, 1H), 3.98-3.88 (m, 1H), 3.77 (s, 3H), 3.60-3.48 (m, 4H), 3.37 (s, 3H), 3.30-3.20 (m, 1H), 1.14 (d, J=6.3, 3H). ¹³C NMR (151 MHz, CDCl₃) δ=160.42, 154.31, 153.55, 151.77, 147.99, 136.78, 130.47, 119.33, 113.63, 113.18, 103.15, 67.63, 55.45, 50.64, 46.88, 29.99, 27.87, 20.83. HRMS (ESI) Calcd for C₁₈H₂₄N₅O₄ [M+H]⁺ 374.1823; Found 374.1832.

### IMB-ZHC-7

(S)-8-((2-hydroxypropyl)amino)-7-(3-methoxybenzyl)-1,3-dimethyl-3,7-dihydro-1H-purine-2,6-dione

### Appearance: White Solid; Yield: 68%.

¹H NMR (600 MHz, CDCl₃) δ=7.29-7.27 (m, 1H), 6.84 (dd, J=11.3, 5.3, 2H), 6.80 (d, J=1.7, 1H), 5.33 (s, 2H), 4.74 (t, J=5.4, 1H), 3.97-3.89 (m, 1H), 3.78 (s, 3H), 3.59-3.52 (m, 4H), 3.39 (s, 3H), 3.27 (ddd, J=13.8, 7.6, 4.9, 1H), 1.16 (d, J=6.3, 3H). ¹³C NMR (151 MHz, CDCl₃) δ=160.40, 154.30, 153.66, 151.79, 148.19, 136.84, 130.44, 139.30, 113.57, 113.16, 103.16, 67.62, 55.43, 50.62, 46.84, 29.91, 27.85, 20.83. HRMS (ESI) Calcd for C₁₈H₂₄N₅O₄ [M+H]⁺ 374.1823; Found 374.1829.

### IMB-ZHC-15

((7-(3-methoxybenzyl)-1,3-dimethyl-2,6-dioxo-2,3,6,7-tetrahydro-1H-purin-8-yl)amino)acetamide

### Appearance: Tan Powder; Yield: 85%.

¹H NMR (500 MHz, CDCl₃) δ=7.84 (s, 1H), 7.34 (d, J=7.8, 1H), 6.89 (dd, J=16.4, 8.9, 4H), 5.35 (s, 2H), 3.82 (d, J=4.9, 5H), 3.61-3.50 (m, 6H). ¹³C NMR (151 MHz, DMSO-d₆) δ=171.26, 159.30, 154.29, 153.13, 150.95, 150.59, 138.54, 129.64, 119.41, 113.52, 112.45, 101.05, 55.01, 45.44, 44.98, 28.41, 28.32.

### IMB-2-26

8-(1H-imidazol-1-yl)-7-(3-methoxybenzyl)-1,3-dimethyl-3,7-dihydro-1H-purine-2,6-dione

### Appearance: White Solid; Yield: 66%.

¹H NMR (500 MHz, CDCl₃) δ=7.86 (s, 1H), 7.24-7.18 (m, 2H), 6.82 (d, J=8.2, 1H), 6.58 (d, J=7.6, 1H), 6.54 (s, 1H), 5.53 (s, 2H), 3.74 (s, 3H), 3.59 (s, 3H), 3.43 (s, 3H). ¹³C NMR (151 MHz, CDCl₃) δ=160.33 (s, 1H), 155.15 (s, 1H), 151.53 (s, 1H), 146.93 (s, 1H), 141.80 (s, 1H), 137.36 (s, 2H), 136.62 (s, 1H), 131.10 (s, 2H), 130.58 (s, 2H), 119.24 (s, 2H), 118.79 (s, 2H), 113.86 (s, 2H), 112.62 (s, 2H), 106.94 (s, 1H), 55.39 (s, 2H), 48.84 (s, 2H), 30.07 (s, 2H), 28.30 (s, 2H). HRMS (ESI) Calcd for C₁₈H₁₉N₆O₃ [M+H]⁺ 367.1519; Found 367.1527.

### IMB-2-31

8-(4-(4-fluorophenyl)piperazin-1-yl)-7-(3-methoxybenzyl)-1,3-dimethyl-3,7-dihydro-1H-purine-2,6-dione

### Appearance: Light Brown Solid; Yield: 82%.

¹H NMR (500 MHz, CDCl₃) δ=7.24 (d, J=7.9, 1H), 6.98 (t, J=8.6, 2H), 6.92-6.89 (m, 2H), 6.82-6.77 (m, 3H), 5.38 (s, 2H), 3.77 (s, 3H), 3.56 (s, 3H), 3.37 (s, 4H), 3.36 (d, J=4.7, 4H), 3.18 (d, J=4.4, 4H). ¹³C NMR (151 MHz, CDCl₃) δ=160.11, 158.45, 156.86, 156.34, 154.86, 151.91, 147.81, 138.39, 130.07, 119.03, 115.88, 115.88, 135.73, 115.73, 112.97, 112.90, 105.27, 55.36, 50.53, 50.08, 48.68, 29.90, 27.99. HRMS (ESI) Calcd for C₂₅H₂₈N₆O₃F [M+H]⁺ 479.2207, Found 479.2203.

### IMB-2-32

Methyl-1-(7-(3-methoxybenzyl)-1,3-dimethyl-2,6-dioxo-2,3,6,7-tetrahydro-1H-purin-8-yl)-1H-indole-5-carboxylate

### Appearance: Light Brown Solid; Yield: 65%.

¹H NMR (500 MHz, CDCl₃) δ=8.05 (s, 1H), 7.94 (d, J=8.3, 1H), 7.71 (d, J=8.3, 1H), 7.26 (s, 1H), 7.10 (t, J=7.9, 1H), 6.77 (d, J=2.9, 1H), 6.72 (d, J=8.2, 1H), 6.50 (d, J=7.5, 1H), 6.36 (s, 1H), 5.45 (s, 2H), 3.92 (s, 3H), 3.62 (s, 3H), 3.59 (s, 3H), 3.47 (s, 3H). ¹³C NMR (151 MHz, CDCl₃) δ=167.45, 159.97, 155.31, 151.65, 147.37, 143.29, 136.81, 136.12, 132.65, 130.16, 129.97, 126.02, 123.33, 121.34, 119.57, 114.05, 113.26, 112.89, 106.87, 106.70, 55.20, 52.29, 49.14, 30.14, 28.32. HRMS (ESI) Calcd for C₂₅H₂₄N₅O₅ [M+H]⁺ 474.1774; Found 474.1770.

### N7-Modified Compounds are as Follows:

### IMB-ZHB-4X

7-(3-chloro-2-fluorobenzyl)-8-((2-hydroxyethyl)amino)-1,3-dimethyl-3,7-dihydro-1H-purine-2,6-dione

### Appearance: White Solid; Yield: 72%.

¹H NMR (500 MHz, DMSO-d₆) δ=7.49 (s, 1H), 7.25 (s, 1H), 7.14 (s, 1H), 6.71 (s, 1H), 5.39 (s, 2H), 4.72 (s, 1H), 3.53 (s, 2H), 3.38 (d, J=1.6, 3H), 3.32 (s, 3H), 3.11 (s, 3H). ¹³C NMR (151 MHz, DMSO-d₆) δ=153.42, 152.41, 151.05, 148.40 135.14, 130.74, 126.91, 126.46, 125.54, 123.96, 101.88, 57.90, 53.40, 44.47, 29.57, 27.33. HRMS (ESI) Calcd for C₁₆H₁₈N₅O₃FCl [M+H]⁺ 382.1082; Found 382.1073.

### IMB-ZHB-13X

7-benzyl-8-((2-hydroxyethyl)amino)-1,3-dimethyl-3,7-dihydro-1H-purine-2,6-dione

### Appearance: White Solid; Yield: 53%.

¹H NMR (500 MHz, DMSO-d₆) δ=7.18 (s, 2H), 5.26 (s, 2H), 4.70 (s, 1H), 3.51 (s, 2H), 3.36 (s, 2H), 3.31 (d, J=6.1, 3H), 3.11 (d, J=6.1, 3H). ¹³C NMR (126 MHz, DMSO-d₆) δ=153.90, 151.00, 148.78, 137.19, 128.51, 128.51, 127.33, 127.02, 127.02, 101.28, 59.74, 45.29, 45.11, 29.33, 27.22. HRMS (ESI) Calcd for C₁₆H₂₀N₅O₃ [M+H]⁺ 330.1566; Found 330.1572

### IMB-68

8-((2-hydroxyethyl)amino)-1,3-dimethyl-7-(2-methylbenzyl)-3,7-dihydro-1H-purine-2,6-dione

### Appearance: White Solid; Yield: 86%.

¹H NMR (500 MHz, DMSO-d₆) δ=7.21 (d, J=7.3, 1H), 7.14 (t, J=7.4, 1H), 7.08 (dd, J=13.7, 6.3, 2H), 6.37 (d, J=7.2, 1H), 5.27 (s, 2H), 4.70 (t, J=5.3, 1H), 3.52 (dd, J=11.2, 5.4, 2H), 3.44-3.37 (m, 5H), 3.10 (s, 3H), 2.33 (s, 3H). ¹³C NMR (151 MHz, DMSO-d₆) δ=153.87, 152.71, 150.97, 148.71, 137.52, 137.04, 128.38, 127.91, 127.53, 123.90, 101.27, 59.68, 45.21, 45.03, 29.29, 27.16, 21.02.

### IMB-79

7-((2-hydroxyethyl)amino)-1,3-dimethyl-7-(3-nitrobenzyl)-3,7-dihydro-1H-purine-2,6-dione

### Appearance: Yellow Solid; Yield: 41%.

¹H NMR (500 MHz, DMSO-d₆) δ=8.22-8.09 (m, 2H), 7.63 (d, J=5.1, 2H), 7.37 (t, J=5.5, 1H), 5.43 (s, 2H), 4.73 (t, J=5.4, 1H), 3.53 (dd, J=11.5, 5.8, 2H), 3.43-3.39 (m, 2H), 3.37 (s, 3H), 3.16 (s, 3H). ¹³C NMR (126 MHz, DMSO-d₆) δ=153.87, 152.83, 150.99, 148.98, 147.80, 139.38, 133.78, 130.25, 122.47, 122.05, 101.11, 59.63, 45.08, 44.77, 29.41, 27.27. HRMS (ESI) Calcd for C₁₆H₁₉N₆O₅ [M+H]⁺ 375.1411; Found 375.1416.

### IMB-80

7-(3-fluorobenzyl)-8-((2-hydroxyethyl)amino)-1,3-dimethyl-3,7-dihydro-1H-purine-2,6-dione

### Appearance: Gray Solid; Yield: 81%.

¹H NMR (500 MHz, DMSO-d₆) δ=7.37 (s, 1H), 7.25 (s, 1H), 7.06 (d, J=10.3, 3H), 5.30 (s, 2H), 4.73 (d, J=3.9, 1H), 3.53 (s, 2H), 3.38 (dd, J=16.4, 4.4, 5H), 3.16 (d, J=3.4, 3H). ¹³C NMR (126 MHz, DMSO-d₆) δ=163.15, 161.21, 153.86, 152.79, 151.01, 148.88, 140.01, 130.61, 123.10, 114.11, 101.17, 59.67, 45.10, 44.85, 29.38, 27.25. Calcd for C₁₆H₁₉N₅O₃F [M+H]⁺ 348.1466; Found 348.1475.

### IMB-81

7-(3-bromobenzyl)-8-((2-hydroxyethyl)amino)-1,3-dimethyl-3,7-dihydro-1H-purine-2,6-dione

### Appearance: Gray Solid; Yield: 78%.

¹H NMR (500 MHz, DMSO-d₆) δ=7.40-7.34 (m, 1H), 7.25 (d, J=3.4, 1H), 7.06 (d, J=10.3, 3H), 5.30 (s, 2H), 4.73 (d, J=3.9, 1H), 3.57-3.51 (m, 2H), 3.42-3.38 (m, 2H), 3.36 (d, J=3.5, 2H), 3.32 (d, J=3.7, 1H), 3.16 (d, J=3.4, 3H). ¹³C NMR (126 MHz, DMSO-d₆) δ=153.84, 152.78, 150.99, 148.88, 139.91, 130.82, 130.29, 129.90, 126.02, 121.72, 101.15, 59.66, 45.08, 44.75, 29.39, 27.26. HRMS (ESI) Calcd for C₁₆H₁₉N₆O₅ [M+H]⁺ 375.1411; Found 375.1422.

### IMB-82

7-(2,3-difluorobenzyl)-8-((2-hydroxyethyl)amino)-1,3-dimethyl-3,7-dihydro-1H-purine-2,6-dione

### Appearance: White Solid; Yield: 89%.

¹H NMR (500 MHz, DMSO-d₆) δ=7.34 (dd, J=17.4, 8.4, 1H), 7.24 (t, J=5.4, 1H), 7.12 (dd, J=12.8, 7.6, 1H), 6.58 (t, J=6.8, 1H), 5.41 (s, 2H), 4.71 (t, J=5.4, 1H), 3.53 (dd, J=11.5, 5.7, 2H), 3.43-3.34 (m, 5H), 3.12 (s. 3H). ¹³C NMR (126 MHz, DMSO-d₆) δ=154.20, 152.66, 151.02, 148.89, 127.12, 127.12, 125.01, 122.34, 116.29, 116.29, 116.22, 101.22, 59.65, 45.12, 29.40, 27.17. HRMS (ESI) Calcd for C₁₆H₁₈N₅O₃F₂ [M+H]⁺ 366.1372; Found 366.1374.

### IMB-83

7-(2,4-difluorobenzyl)-8-((2-hydroxyethyl)amino)-1,3-dimethyl-3,7-dihydro-1H-purine-2,6-dione

### Appearance: Gray Solid; Yield: 84%.

¹H NMR (500 MHz, DMSO-d₆) δ=7.28 (t, J=10.0, 1H), 7.20 (d, J=5.3, 1H), 7.00 (t, J=8.3, 1H), 6.85 (dd, J=14.9, 8.3, 1H), 5.33 (s, 2H), 4.71 (t, J=5.4, 1H), 3.39 (d, J=11.4, 2H), 3.31 (s, 3H), 3 12 (s, 2H), 2.50 (s, 3H). ¹³C NMR (101 MHz, DMSO-d₆) δ=160.53, 158.26, 154.18, 152.66, 151.03, 148.91, 128.70, 120.67, 111.53, 103.93, 101.21, 59.68, 45.11, 29.38, 27.17. HRMS (ESI) Calcd for C₁₆H₁₈N₅O₃F₂ [M+H]⁺ 366.1372; Found 366.1378.

### IMB-84

7-(4-(tert-butyl)benzyl)-8-((2-hydroxyethyl)amino)-1,3-dimethyl-3,7-dihydro-1H-purine-2,6-dione

### Appearance: Brown Solid; Yield: 58%.

¹H NMR (500 MHz, DMSO-d₆) δ=7.33 (d, J=8.1, 2H), 7.21 (d, J=10.7, 1H), 7.15 (d, J=8.0, 2H), 5.25 (s, 2H), 4.73 (t, J=5.4, 1H), 3.55 (dd, J=11.6, 5.8, 2H), 3.42-3.38 (m, 2H), 3.35 (s, 2H), 3.32 (s, 1H), 3.15 (s, 3H), 1.24 (s, 9H). ¹³C NMR (101 MHz, DMSO-d₆) δ=154.34, 153.20, 151.44, 150.16, 149.23, 134.66, 127.18, 125.72, 101.70, 60.16, 45.54, 45.38, 34.65, 31.54, 29.77, 27.66. HRMS (ESI) Calcd for C₂₀H₂₈N₅O₃ [M+H]⁺ 386.2187; Found 386.2193.

### IMB-85

7-(4-butylbenzyl)-8-((2-hydroxyethyl)amino)-1,3-dimethyl-3,7-dihydro-1H-purine-2,6-dione

### Appearance: Light Yellow Solid; Yield: 85%.

¹H NMR (500 MHz, DMSO-d₆) δ=7.15 (d, J=29.4, 5H), 5.25 (s, 2H), 4.72 (s, 1H), 3.54 (s, 2H), 3.37 (d, J=19.8, 5H), 3.31 (s, 2H), 3.15 (s, 3H), 1.50 (s, 2H), 3.28 (s, 2H), 0.87 (s, 3H). ¹³C NMR (101 MHz, DMSO-d₆) δ=153.88, 152.77, 151.01, 148.79, 141.47, 134.39, 128.40, 128.40, 127.03, 127.03, 101.28, 59.72, 45,08. 34.47, 33.15, 29.34, 27.23, 21.78, 13.77. HRMS (ESI) Calcd for C₂₀H₂₈N₅O₃ [M+H]⁺ 386.2187; Found 386.2195.

### IMB-88

7-(3,5-difluorobenzyl)-8-((2-hydroxyethyl)amino)-1,3-dimethyl-3,7-dihydro-1H-purine-2,6-dione

### Appearance: White Solid; Yield: 91%.

¹H NMR (500 MHz, DMSO-d₆) δ=7.22 (d, J=8.5, 3H), 6.87 (s, 2H), 5.21 (s, 2H), 4.73 (d, J=3.8, 1H), 3.70 (d, J=3.5, 3H), 3.54 (s, 2H), 3.38 (d, J=14.4, 2H), 3.36 (s, 4H), 3.17 (d, J=3.3, 2H). ¹³C NMR (101 MHz, DMSO-d₆) δ=158.62, 153.79, 152.80, 151.01, 148.82, 129.11, 128.74, 128.74, 113.88, 113.88, 101.19, 59.75, 55.07, 45.09, 44.74, 29.33, 27.25. HRMS (ESI) Calcd for C₁₇H₂₂N₅O₄ [M+H]⁺ 360.1666; Found 360.1575.

### IMB-92

8-((2-hydroxyethyl)amino)-1,3-dimethyl-7-(3-(trifluoromethoxy)benzyl)-3,7-dihydro-1H-purine-2,6-dione

### Appearance: White Solid; Yield: 91%.

¹H NMR (500 MHz, DMSO-d₆) δ=7.45 (t, J=7.9, 1H), 7.34-7.24 (m, 3H), 7.21 (d, J=7.5, 1H), 5.34 (s, 2H), 4.73 (t, J=5.3, 1H), 3.55-3.49 (m, 2H), 3.39 (d, J=5.7, 2H), 3.36 (s, 3H), 3.15 (s, 3H). ¹³C NMR (101 MHz, CDCl₃) δ=159.09, 158.02, 156.21, 154.14, 153.65, 145.03, 135.83, 135.83, 131.21, 125.05, 124.90, 106.29, 64.85, 50.25, 50.01, 34.56, 32.43. HRMS (ESI) Calcd for C₁₇H₁₉N₅O₄F₃ [M+H]⁺ 414.1384; Found 414.1380.

### IMB-93

7-(2,6-difluorobenzyl)-8-((2-hydroxyethyl)amino)-1,3-dimethyl-3,7-dihydro-1H-purine-2,6-dione

### Appearance: Gray Solid; Yield: 73%.

¹H NMR (500 MHz, DMSO-d₆) δ=7.43-7.30 (m, 1H), 7.12 (t, J=5.4, 1H), 7.04 (t, J=8.1, 2H), 5.35 (s, 2H), 4.74 (t, J=5.4, 1H), 3.55 (dd, J=11.6, 5.8, 2H), 3.46-3.39 (m, 2H), 3.35 (s, 1H), 3.31 (s, 3H), 3.08 (s, 2H). ¹³C NMR (101 MHz, DMSO-d₆) δ=161.93 (d, J=8.1, 1H), 159.46 (d, J=8.0, 1H), 154.48, 152.53, 151.02, 148.76, 129.96, 112.77, 111.94, 111.51, 101.51, 59.85, 45.20, 36.54, 29.34, 27.18. HRMS (ESI) Calcd for C₁₆H₁₈N₅O₃F₂ [M+H]⁺ 366.1372; Found 366.1365.

### IMB-2-2

7-(4-fluorobenzyl)-8-((2-hydroxyethyl)amino)-1,3-dimethyl-3,7-dihydro-1H-purine-2,6-dione

### Appearance: White Solid; Yield: 90%.

¹H NMR (500 MHz, DMSO-d₆) δ=7.29 (d, J=25.5, 2H), 7.17 (s, 2H), 5.30 (s, 2H), 4.75 (t, J=5.4, 1H), 3.55 (dd, J=10.3, 5.2, 2H), 3.41 (dd, J=9.4, 3.7, 2H), 3.38 (s, 3H), 3.18 (s, 3H). ¹³C NMR (101 MHz, CDCl₃) δ=165.49, 159.02, 158.01, 156.21, 154.09, 138.52, 134.53, 120.51, 106.35, 64.88, 50.27, 49.81, 34.56, 32.45. HRMS (ESI) Calcd for C₁₆H₁₉N₅O₃F [M+H]⁺ 348.1466; Found 348.1459.

### IMB-2-3

8-((2-hydroxyethyl)amino)-1,3-dimethyl-7-(4-(trifluoromethoxy) benzyl)-3,7-dihydro-1H-purine-2,6-dione

### Appearance: White Solid; Yield: 84%.

¹H NMR (500 MHz, DMSO-d₆) δ=7.33 (s, 4H), 7.28 (t, J=4.6, 1H), 5.33 (s, 2H), 4.73 (t, J=5.1, 1H), 3.53 (dd, J=11.1, 5.1, 2H), 3.42-3.38 (m, 2H), 3.36 (s, 3H), 3.15 (s, 3H). ¹³C NMR (101 MHz, CDC₃) δ=159.08, 158.01, 156.22, 154.30, 152.75, 152.75, 141.83, 134.15, 134.15, 126.40, 126.40, 106.37, 64.85, 50.28, 49.85, 34.57, 32.44. HRMS (ESI) Calcd for C₁₇H₁₉N₅O₄F₃ [M+H]⁺ 414.1384; Found 414.1375.

### IMB-2-5

7-(3-chlorobenzyl)-8-((2-hydroxyethyl)amino)-1,3-dimethyl-3,7-dihydro-1H-purine-2,6-dione

### Appearance: Brown Solid; Yield: 88%.

¹H NMR (500 MHz, DMSO-d₆) δ=7.38-7.31 (m, 3H), 7.26 (s, 1H), 7.15 (d, J=6.9, 1H), 5.30 (s, 2H), 4.73 (t, J=5.4, 1H), 3.53 (t, J=5.6, 2H), 3.53 (t, J=5.6, 2H), 3.43-3.37 (m, 2H), 3.31 (s, 4H), 3.16 (s, 2H). ¹³C NMR (151 MHz, DMSO-d₆) δ=153.92, 152.85, 151.07, 148.93, 139.65, 133.15, 130.54 (, 127.44, 127.01, 125.71, 101.24, 59.72, 45.13, 44.87, 29.41, 27.29. HRMS (ESI) Calcd for C₁₆H₁₉N₅O₃Cl [M+H]⁺ 364.1171; Found 364.1163.

### IMB-2-6

7-(2-bromobenzyl)-8-((2-hydroxyethyl)amino)-1,3-dimethyl-3,7-dihydro-1H-purine-2,6-dione

### Appearance: Milk White Solid; Yield: 92%.

¹H NMR (500 MHz, DMSO-d₆) δ=7.67 (d, J=7.8, 1H), 7.32-7.26 (m, 2H), 7.22 (t, J=7.6, 1H), 6.44 (d, J=7.3, 1H), 5.32 (s, 2H), 4.69 (t, J=5.4, 1H), 3.52 (d, J=5.7, 2H), 3.41 (s, 5H), 3.10 (s, 3H). ¹³C NMR (101 MHz DMSO-d₆) δ=154.33, 152.66, 151.08, 148.92, 136.06, 132.60, 128.98, 128.12, 125.81, 121.23, 101.14, 59.68, 46.33, 45.11, 29.45, 27.16. HRMS (ESI) Calcd for C₁₆H₁₉N₅O₃Br [M+H]⁺ 408.0666; Found 408.0657.

### IMB-2-8

7-(4-(difluoromethoxy)benzyl)-8-((2-hydroxyethyl)amino)-1,3-dimethyl-3,7-dihydro-1H-purine-2,6-dione

### Appearance: White Solid; Yield: 94%.

¹H NMR (500 MHz, DMSO-d₆) δ=7.29 (s, 3H), 7.12 (d, J=8.8, 2H), 5.28 (s, 2H), 3.54 (s, 2H), 3.39 (s, 2H), 3.35 (d, J=7.3, 3H), 3.16 (s, 3H). ¹³C NMR (151 MHz, DMSO-d₆) δ=153.80, 152.75, 150.96, 150.15, 148.83, 134.11, 128.85, 128.85, 118.85, 118.85, 116.30, 101.11, 59.65, 45.06, 44.61, 29.28, 27.18. HRMS (ESI) Calcd for C₁₇H₂₀N₅O₄F₂ [M+H]⁺+396.1478; Found 396.1469.

### IMB-2-14

8-bromo-7-(4-chlorobenzyl)-1,3-dimethyl-3,7-dihydro-1H-purine-2,6-dione

### Appearance: White Solid; Yield: 96%.

¹H NMR (500 MHz, DMSO-d₆) δ=7.39 (s, 2H), 7.25 (s, 3H), 5.29 (s, 2H), 4.74-4.71 (m, 1H), 3.53 (s, 2H), 3.35 (d, J=4.4, 5H), 3.15 (s, 3H). HRMS (ESI) Calcd for C₁₆H₁₉N₅O₃Cl [M+H]⁺ 364.1176; Found 364.1172.

### IMB-3-19

((2-hydroxyethyl)amino)-1,3-dimethyl-7-((tetrahydro-2H-pyran-4-yl)methyl)-3,7-dihydro-1H-purine-2,6-dione

### Appearance: White Solid; Yield: 76%.

¹H NMR (500 MHz, DMSO-d₆) δ=7.00 (s, 1H), 5.75 (s, 1H), 4.73 (s, 1H), 3.90 (d, J=5.0, 2H) 3.81 (d, J=9.8, 2H), 3.54 (s, 2H), 3.39 (s, 2H), 3.34 (s, 4H), 3.26-3.08 (m, 5H), 1.99 (s, 1H), 1.32 (dd, J=38.0, 11.2, 4H). ¹³C NMR (126 MHz, DMSO-d₆) δ=153.82, 152.57, 150.98, 148.47, 101.73, 66.58, 66.58, 59.77, 47.29, 45.06, 34.92, 29.38, 29.38, 29.28, 27.22. HRMS (ESI) Calcd for C₁₅H₂₄N₅O₄ [M+H]⁺ 338.1828; Found 338.1824.

### IMB-4-3

8-((2-hydroxyethyl)amino)-1,3-dimethyl-7-(3-methylbenzyl)-3,7-dihydro-1H-purine-2,6-dione

### Appearance: White Solid; Yield: 83%.

¹H NMR (500 MHz, CDCl₃) δ=7.24 (d, J=8.0, 1H), 7.12 (d, J=7.6, 1H), 7.04 (d, J=12.0, 2H), 5.33 (s, 2H), 4.50 (s, 1H), 3.76-3.69 (m, 2H), 3.53 (d, J=6.7, 5H), 3.40 (s, 3H), 2.33 (s, 3H). ¹³C NMR (151 MHz, CDCl₃) δ=154.29, 153.73, 151.81, 148.28, 139.21, 135.22, 129.20, 129.17, 127.74, 124.09, 103.29, 62.69, 46.85, 46.00, 29.84, 27.85, 21.52. HRMS (ESI) Calcd for C₁₇H₂₂N₅O₃ [M+H]⁺ 344.1723; Found 344.1701.

### IMB-4-6

ethyl3-((8-((2-hydroxyethyl)amino)-1,3-dimethyl-2,6-dioxo-1,2,3,6-tetrahydro-7H-purin-7-yl)methyl)benzoate

### Appearance: White Solid; Yield: 88%.

¹H NMR (500 MHz, CDCl₃) δ=8.01-7.91 (m, 2H), 7.51 (d, J=7.2, 1H), 7.45 (d, J=7.6, 1H), 5.40 (s, 2H), 4.66 (s, 1H), 4.38 (q, J=7.2. 2H), 3.78 (t, J=3.8, 2H), 3.59 (dd, J=9.9, 5.7, 2H), 3.52 (s, 3H), 3.38 (s, 3H), 3.39 (t, J=7.1, 3H). ¹³C NMR (151 MHz, CDCl₃) δ=166.58, 154.25, 153.63, 151.80, 148.65, 136.08, 131.92, 131.19, 129.48, 129.38, 128.15, 102.95, 62.27, 61.54, 46.35, 45.80, 29.85, 27.83, 14.37. HRMS (ESI) Calcd for C₁₉H₂₄N₅O₅ [M+H]⁺ 402.1777; Found 402.1775.

### IMB-4-12

8-((2-hydroxyethyl)amino)-1,3-dimethyl-7-(thiophen-3-ylmethyl)-3,7-dihydro-1H-purine-2,6-dione

### Appearance: Light Brown Solid; Yield: 72%.

¹H NMR (500 MHz, CDCl₃) δ=7.41-7.36 (m, 1H), 7.27 (s, 1H), 7.08 (d, J=4.9, 1H), 5.40 (s, 2H), 4.65 (s, 1H), 3.84-3.76 (m, 2H), 3.61 (dd, J=9.5, 5.3, 2H), 3.55 (s, 3H), 3.43 (s, 3H). ¹³C NMR (151 MHz, DMSO-d₆) δ=153.56, 152.77, 150.97, 148.75, 137.56, 127.41, 126.59, 123.21, 101.02, 59.70, 45.03, 40.85, 29.27, 27.19. HRMS (ESI) Calcd for C₁₄H₁₈N₅O₃S [M+H]⁺ 336.1130; Found 336.1124.

### IMB-4-13

8-((2-hydroxyethyl)amino)-7-(3-methoxyphenethyl)-1,3-dimethyl-3,7-dihydro-1H-purine-2,6-dione

### Appearance: White Solid; Yield: 55%.

¹H NMR (500 MHz, CDCl₃) δ=7.27 (d, J=8.0, 1H), 6.85 (d, J=6.4, 1H), 6.75 (d, J=7.4, 1H), 6.66 (s, 1H), 4.27 (t, J=6.2, 2H), 3.80 (s, 3H), 3.75 (s, 1H), 3.61-3.56 (m, 2H), 3.53 (s, 3H), 3.45 (s, 3H), 3.27 (d, J=9.5, 5.2, 2H), 3.10 (t, J=6.1, 2H). ¹³C NMR (151 MHz, DMSO-d₆) δ=159.22, 153.46, 152.66, 151.00, 348.61, 139.48, 129.24, 121.34, 114.50, 111.84, 101.20, 59.75, 54.89, 44.95, 43.59, 35.11, 29.24, 27.21. HRMS (ESI) Calcd for C₁₈H₂₄N₅O₄ [M+H]⁺ 374.1828; Found 374.1821.

### N1-Modified Compounds are as Follows:

### IMB-3-6

8-((2-hydroxyethyl)amino)-7-(3-methoxybenzyl)-3-methyl-1-((tetrahydro-2H-pyran-4-yl)methyl)-3,7-dihydro-1H-purine-2,6-dione

### Appearance: White Solid; Yield: 77%.

¹H NMR (500 MHz, CDCl₃) δ=7.28 (d, J=7.9, 1H), 6.87-6.79 (m, 3H), 5.35 (s, 2H), 4.79 (s, 1H), 3.93 (t, J=11.6, 4H), 3.78 (s, 3H), 3.77-3.73 (m, 2H), 3.60 (s, 2H), 3.54 (s, 3H), 3.34 (t, J=11.6, 2H), 2.61 (s, 1H), 2.07 (ddd, J=11.4, 7.4, 4.0, 1H), 1.56 (d, J=12.4, 2H), 1.51-1.42 (m, 2H). ¹³C NMR (151 MHz, CDCl₃) δ=160.40, 154.40, 153.78, 151.83, 148.43, 136.83, 130.44, 119.30, 113.58, 113.20, 103.19, 67.82, 67.82, 62.74, 55.40, 46.86, 46.26, 46.01, 34.43, 30.91, 30.91, 29.84. HRMS (ESI) Calcd for C₂₂H₃₀N₅O₅ [M+H]⁺ 444.2247; Found 444.2241.

### IMB-3-13

1-(4-fluorobenzyl)-8-((2-hydroxyethyl)amino)-7-(3-methoxybenzyl)-3-methyl-3,7-dihydro-1H-purine-2,6-dione

### Appearance: White Solid; Yield: 18%.

¹H NMR (600 MHz, CDCl₃) δ=7.48-7.44 (m, 2H), 7.28 (d, J=7.9, 1H), 6.87-6.77 (m, 5H), 5.33 (s, 2H), 5.30 (s, 1H), 5.12 (s, 2H), 4.59 (s, 1H), 3.76 (s, 3H), 3.74-3.71 (m, 2H), 3.53 (dd, J=9.7, 5.0, 2H), 3.51-3.49 (m, 3H). ¹³C NMR (151 MHz, CDCl₃) δ=160.41, 159.01, 154.16, 153.72, 151.69, 148.39, 136.80, 130.47, 130.47, 130.42, 130.25, 119.28, 113.80, 113.80, 113.77, 113.03, 103.33, 62.76, 55.38, 46.88, 46.02, 43.74, 29.84.

### IMB-3-15

8-((2-hydroxyethyl)amino)-7-(3-methoxybenzyl)-1-(4-methoxybenzyl)-3-methyl-3,7-dihydro-1H-purine-2,6-dione

### Appearance: White Solid; Yield: 20%.

¹H NMR (500 MHz, CDCl₃) δ=7.16 (dt, J=20.1, 8.0, 2H), 6.98-6.92 (m, 2H), 6.80-6.68 (m, 4H), 5.46 (s, 1H), 5.25 (s, 2H), 5.09 (s, 2H), 3.71 (s, 3H), 3.68 (d, J=9.1, 5H), 3.49 (d, J=3.8, 2H), 3.44 (s, 3H).

### IMB-3-16

8-((2-hydroxyethyl)amino)-7-(3-methoxybenzyl)-3-methyl-1-(pyridin-4-ylmethyl)-3,7-dihydro-1H-purine-2,6-dione

### Appearance: White Solid; Yield: 20%.

¹H NMR (500 MHz, CDCl₃) δ=7.40 (d, J=7.5, 2H), 7.25-7.15 (m, 4H), 6.78 (dd, J=13.4, 8.1, 3H), 5.45 (s, 1H), 5.26 (s, 2H), 5.12 (s, 2H), 3.68 (d, J=9.4, 5H), 3.50 (s, 2H), 3.45 (s, 3H). ¹³C NMR (151 MHz, CDCl₃) δ=160.40, 154.11, 153.76, 151.69, 148.47, 137.94, 136.80, 330.40, 128.76, 128.76, 128.46, 128.46, 127.42, 119.28, 113.78, 113.03, 103.27, 62.72, 55.39, 46.87, 46.00, 44.31, 29.88. HRMS (ESI) Calcd for C₂₂H₂₅N₅O₄ [M+H]⁺ 437.1937; Found 437.1931

### IMB-3-30

8-bromo-1-ethyl-7-(3-methoxybenzyl)-3-methyl-3,7-dihydro-1H-purine-2,6-dione

### Appearance: White Solid; Yield: 45%.

¹H NMR (500 MHz, CDCl₃) δ=6.94 (d, J=12.0, 2H), 6.85 (d, J=8.2, 1H), 5.53 (s, 2H), 4.07 (q, J=7.0, 2H), 3.78 (s, 2H), 3.55 (s, 3H), 1.24 (t, J=7.0, 3H). ¹³C NMR (151 MHz, CDCl₃) δ=160.07, 154.15, 151.03, 148.52, 136.64, 130.11, 127.88, 120.19, 113.81, 109.37, 55.38, 50.25, 36.87, 29.90, 13.36. HRMS (ESI) Calcd for C₁₆H₁₈N₄O₃Br [M+H]⁺ 393.0562; Found 393.0570.

### IMB-3-45

1-(but-3-yn-1-yl)-8-((2-hydroxyethyl)amino)-7-(3-methoxybenzyl)-3-methyl-3,7-dihydro-1H-purine-2,6-dione

### Appearance: Yellow Solid; Yield: 28%.

¹H NMR (500 MHz, CDCl₃) δ=7.33-7.27 (m, 1H), 6.83 (dd, J=15.7, 7.6, 3H), 5.32 (s, 2H), 4.57 (s, 1H), 4.21 (t, J=6.2, 2H), 3.78 (d, J=1.9, 3H), 3.75 (s, 2H), 3.55 (s, 2H), 3.51 (d, J=1.9, 3H), 2.886 (s, J=2.886, 1H), 1.96 (d, J=2.2, 1H), 1.26 (s, 1H). ¹³C NMR (151 MHz, CDCl₃) δ=160.38, 153.84, 153.76, 151.42, 148.51, 136.84, 130.41, 119.31, 113.49, 113.28, 103.16, 81.26, 69.78, 62.71, 55.42, 46.84, 46.00, 39.43, 29.80, 17.91. HRMS (ESI) Calcd for C₂₀H₂₄N₅O₄ [M+H]⁺ 398.1828; Found 398.1826.

### IMB-3-46

ethyl-8-((2-hydroxyethyl)amino)-7-(3-methoxybenzyl)-3-methyl-3,7-dihydro-1H-purine-2,6-dione

### Appearance: White Solid; Yield: 54%.

¹H NMR (500 MHz, CDCl₃) δ=7.28 (d, J=7.9, 1H). 6.83 (dd. J =: 15.4, 7.7, 3H), 5.33 (s, 2H), 4.55 (s, 1H), 4.07 (q, J=6.8, 2H), 3.78 (s, 3H), 3.74 (s, 2H), 3.53 (dd, J=9,6, 5.3, 2H), 3.50 (s, 3H), 3.00 (s, 1H), 1.24 (t, J=6.9, 3H). ¹³C NMR (151 MHz, CDCl₃) δ=160.39, 154.05, 153.66, 151.42, 148.24, 136.89, 130.43, 119.28, 113.57, 113.13, 103.35, 62.80, 55.41, 46.83, 46.05, 36.35, 29.75, 13.56. HRMS (ESI) Calcd for C₁₈H₂₄N₅O₄ [M+H]⁺ 374.1828; Found 374.1807

### IMB-3-47

8-((2-hydroxyethyl)amino)-7-(3-methoxybenzyl)-3-methyl-1-propyl-3,7-dihydro-1H-purine-2,6-dione

### Appearance: White Solid; Yield: 36%.

¹H NMR (500 MHz, CDCl₃) δ=7.28 (d, J=7.8, 31), 6.83 (dd, J=14.7, 7.0, 3H), 5.33 (s, 2H), 4.58 (s, 1H), 4.00-3.92 (m, 2H), 3.78 (s, 3H), 3.74 (s, 2H), 3.54 (dd, J=9.6, 5.2, 2H), 3.50 (s, 3H), 3.04 (s, 1H), 1.73-1.64 (m, 2H), 0.95 (t, J=7.4, 3H). ¹³C NMR (151 MHz, CDCl₃) δ=160.37, 154.24, 153.67, 151.57, 148.25, 136.91, 130.40, 119.27, 113.58, 113.09, 103.29, 62.78, 55.40, 46.80, 46.04, 42.78, 29.77, 21.55, 11.47. HRMS (ESI) Calcd for C₁₉H₂₆N₅O₄ [M +H]⁺ 388.1985; Found 388.1979.

### IMB-3-57

tert-butyl(2-(8-((2-hydroxyethyl)amino)-7-(3-methoxybenzyl)-3-methyl-2,6-dioxo-2,3,6,7-tetrahydro-1H-purin-1-yl)ethyl)carbamate

### Appearance: White Crystal; Yield: 12%.

¹H NMR (500 MHz, CDCl₃) δ=7.36-7.30 (m, 1H), 6.87 (t, J=7.4, 2H), 6.83 (s, 1H), 5.34 (d, J=3.9, 2H), 4.21 (s, 2H), 3.82 (s, 3H), 3.80-3.74 (m, 2H), 3.58 (dd, J=9.6, 5.2, 2H), 3.54 (s, 3H), 3.46 (s, 2H), 1.64 (s, 1H), 1.42 (s, 9H). ¹³C NMR (151 MHz, CDCl₃) δ=160.27, 156.17, 154.23, 153.79, 151.64, 148.51, 136.78, 130.32, 119.19, 113.49, 313.04, 103.04, 78.94, 62.48, 55.30, 46.71, 45.86, 38.23, 37.26, 29.75, 28.49, 28.49, 28.49.

### IMB-3-58

tert-butyl(3-(8-((2-hydroxyethyl)amino)-7-(3-methoxybenzyl)-3-methyl-2,6-dioxo-2,3,6,7-tetrahydro-1H-purin-1-yl)propyl)carbamate

### Appearance: White Crystal; Yield: 40%.

¹H NMR (500 MHz, CDCl₃) δ=7.28 (d, J=6.8, 1H), 6.86-6.81 (m, 2H), 6.79 (s, 1H), 5.31 (s, 2H0, 4.68 (t, J=5.4, 1H), 4.07 (t, J=6.1, 2H), 3.78 (s, 3H), 3.76-3.71 (m, 2H), 3.54 (dd, J=9.7, 5.2, 2H), 3.50 (s, 3H), 3.09 (d, J=5.3, 2H), 1.87-1.79 (m, 2H), 1.43 (s, 9H). ¹³C NMR (151 MHz, CDCl₃) δ=360.22, 156.15, 354.18, 153.80, 151.63, 148.53, 136.78, 130.27, 119.35, 113.43, 113.00, 102.99, 78.93, 62.40, 55.26, 46.65, 45.82, 38.20, 37.25, 29.71, 28.46, 28.46, 28.46, 28.36. HRMS (ESI) Calcd for C₂₄H₃₅N₆O₆ [M+H]⁺ 503.2618, Found 503.2603.

### IMB-3-71

allyl-8-((2-hydroxyethyl)amino)-7-(3-methoxybenzyl)-3-methyl-3,7-dihydro-1H-purine-2,6-dione

### Appearance: Yellow Solid; Yield: 52%.

¹H NMR (500 MHz, CDCl₃) δ=7.24 (d, J=7.9, 1H), 6.80 (dd, J=15.9, 7.5, 3H) 5.91 (dq, J=10.7, 5.6, 1H), 5.29 (d, J=3.8, 2H), 5.22 (d, J=17.2, 1H), 5.14 (d, J=10.2, 1H), 4.70 (t, J=5.3, 1H), 4.59 (d, J=5.5, 2H), 3.75 (s, 3H), 3.72 (dd, J=13.6, 8.9, 2H), 3.52 (dd, J=9.8, 5.2, 3H). ¹³C NMR (151 MHz, CDCl₃) δ=160.38, 353.84, 153.78, 151.41, 148.51, 136.83, 132.92, 130.41, 119.27, 117.13, 113.62, 113.10, 103.22, 62.72, 55.41, 46.84, 46.00, 43.18, 29.80. HRMS (ESI) Calcd for C₁₉H₂₄N₅O₄ [M+H]⁺ 386.1828; Found 386.1816.

### IMB-3-72

8-((2-hydroxyethyl)amino)-7-(3-methoxybenzyl)-3-methyl-1-(prop-2-yn-1-yl)-3,7-dihydro-1H-purine-2,6-dione

### Appearance: White Solid; Yield: 16%.

¹H NMR (500 MHz, CDCl₃) δ=7.25 (d, J=4.3, 1H), 6.81 (dd, J=15.1, 7.3, 3H), 5.30 (s, 2H), 5.29 (s, 1H), 4.76 (d, J=2.2, 2H), 4.66 (t, J=5.3, 1H), 3.77 (s, 3H), 3.75-3.68 (m, 2H), 3.54 (dd, J=9.5, 4.9, 2H), 3.48 (s, 3H), 2.15 (t, J=2.1, 1H). ¹³C NMR (151 MHz, CDCl₃) δ=160.44, 153.87, 153.11, 151.04, 148.73, 136.67, 130.49, 119.34, 113.75, 113.17, 103.16, 79.32, 70.29, 62.67, 55.46, 46.99, 45.98, 30.34, 29.91. HRMS (ESI) Calcd for C₁₉H₂₂N₅O₄ [M+H]384.1672; Found 384.1662.

### N3-Modified Compound is as Follows:

### IMB-3-81

benzyl-8-((2-hydroxyethyl)amino)-7-(3-methoxybenzyl)-1-methyl-3,7-dihydro-1H-purine-2,6-dione

### Appearance: White Solid; Yield: 98%.

¹H NMR (500 MHz, CDCl₃) δ=7.51 (d, J=7.1, 2H), 7.31 (d, J=8.0, 3H), 6.87-6.82 (m, 2H), 6.81 (s, 1H), 5.32 (s, 2H), 5.21 (s, 2H), 3.78 (s, 3H), 3.76 (dd, J=7.1, 2.8, 2H), 3.55 (dd, J=9.5, 4.5, 2H), 3.39 (s, 3H). ¹³C NMR (151 MHz, CDCl₃) δ=160.43, 154.38, 153.61, 153.59, 147.92, 136.83, 136.81, 130.51, 128.97, 128.97, 128.63, 128.63, 127.92, 119.40, 113.64, 113.25, 103.39, 62.70, 55.45, 46.95, 46.75, 46.05, 27.91. HRMS (ESI) Calcd for C₂₃H₂₆N₅O₄ [M+H]⁺ 436.1985; Found 436.1987.

### EXAMPLE 2. DETERMINATION OF THE ANTI-CORONAVIRUS HCoV-229E ACTIVITY OF IMB-C5 SERIES COMPOUNDS BY CYTOPATHIC EFFECT (CPE) ASSAY

(1) The experiments were performed in Huh7 and Huh7.5 cells. The cells were seeded into 96-well culture plates and cultured in 5% CO₂ at 37° C. for about 24 h;
(2) The IMB-C5 series compounds were diluted in DMEM medium containing 2% FBS and 1% penicillin-streptomycin at a dilution of 3 to obtain samples of 8 doses;
(3) The cells were infected with HCoV-229E at 100 TCID₅₀ and added with samples of different dilutions. At the same time, cell control wells and virus control wells were set. The cells were cultured in 5% CO₂ at 35° C. for about 48 h. When cytopathic effect (CPE) in the virus control group reached 4+, cytopathic effect in each group was observed. Reed-Muench method was used to calculate the half toxic concentration (TC₅₀) of the samples to cells and the half inhibitory concentration (EC₅₀) of the samples to viruses, and the selection index (SI=TC₅₀/EC₅₀) was calculated.

CPE evaluation criteria: 4+(cell death percentage of 75% to 100%), 3+(cell death percentage of 50% to 75%), 2+(cell death percentage of 25% to 50%), 1+(cell death percentage of 0 to 25%) and 0+(all cells survive), according to cell death percentage.

The results are as shown in Table 1 and Table 2. IMB-C5 series compounds exhibited a good activity of inhibiting HCoV-229E in Huh7 and Huh7.5 cells. Furthermore, most compounds showed an activity superior to that of the positive control Ribavirin (RBV) and comparable or superior to that of the commercially available RdRp inhibitor Molnupiravir (MNP). Among them, compound IMB-85 had the best activity, and the EC₅₀ of IMB-85 for inhibiting HCoV-229E in Huh7 and Huh7.5 cells was 0.09 μM and 4.05 μM, respectively, superior to that of the positive control and other IMB-C5 homologous compounds.

Compounds IMB-ZH-2, IMB-ZH-11, IMB-ZH-12, IMB-ZHC-2, IMB-ZHC-15, IMB-2-26, IMB-2-31, IMB-2-32, IMB-3-19, IMB-4-6, IMB-4-12, IMB-4-13, IMB-ZHB-4x, IMB-2-3, IMB-2-8, IMB-68, IMB-82, IMB-83, IMB-84, IMB-85, IMB-92, IMB-93, IMB-3-6, IMB-3-13, IMB-3-15, IMB-3-16, IMB-3-30, IMB-3-45, IMB-3-46, IMB-3-47, IMB-3-57, IMB-3-58, IMB-3-71, IMB-3-72 and IMB-3-81 in the table are the compounds with new structures.

TABLE 1
The anti-HCoV-229E activity of IMB-C5 series compounds in Huh7 cells
determined by CPE assay

| | | HCoV-229E | | |
|---|---|---|---|---|
| Compound | Compound | TC₅₀ | EC₅₀ | |
| information | Structure | (μM) | (μM) | SI |
| IMB-CS | 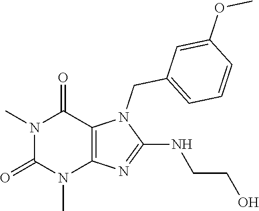 | >139.12 | 6.57 | >21.19 |
| IMB-ZH-2 (C8 modification) | 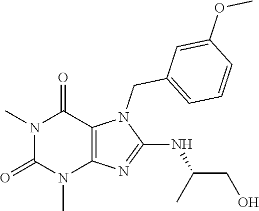 | >133.98 | 44.67 | >3 |
| IMB-ZH-6 (C8 modification) | 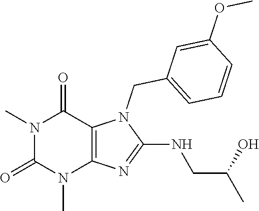 | >133.98 | 34.65 | >3.87 |
| IMB-ZH-10 (C8 modification) | 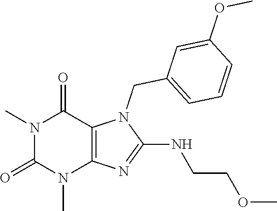 | >133.98 | 8.60 | >15.58 |
| IMB-ZH-11 (C8 modification) | 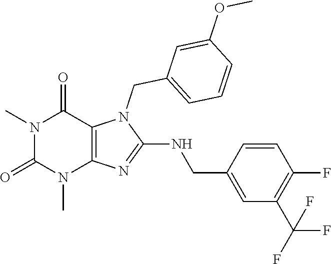 [SMILES: COc1cccc(Cn2c(NCc3ccc(F)c(C(F)(F)F)c3)nc3c2c(=O)n(C)c(=O)n3C)c1] | >101.79 | 20.99 | >4.85 |
| IMB-ZH-12 (C8 modification) | 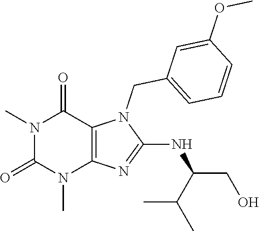 | >124.63 | 59.92 | >2.08 |
| IMB-ZHC-2 (CS modification) | 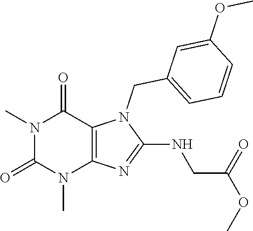 | >129.17 | 28.67 | >4.5 |
| IMB-ZHC-6 (C8 modification) | 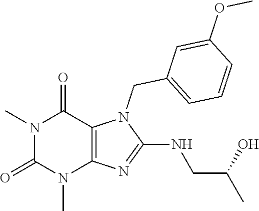 | >133.98 | 12.62 | >10.61 |
| IMB-ZHC-7 (C8 modification) | 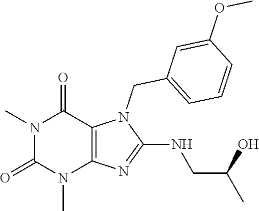 | >133.98 | 19.19 | >6.98 |
| IMB-ZHC-15 (C8 modification) | 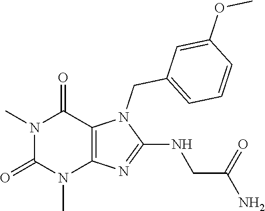 | >134.34 | 44.79 | >3 |
| IMB-2-26 (CS modification) | 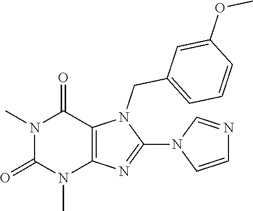 | >200 | 38.49 | >5.20 |
| IMB-2-31 (C8 modification) | 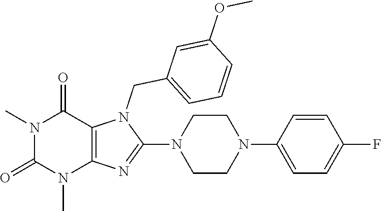 [SMILES: COc1cccc(Cn2c(N3CCN(c4ccc(F)cc4)CC3)nc3c2c(=O)n(C)c(=O)n3C)c1] | >200 | 38.49 | >5.20 |
| IMB-2-32 (C8 modification) | 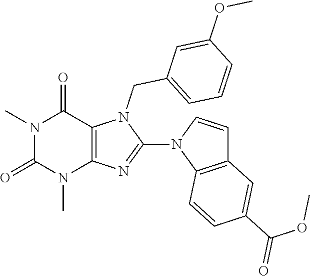 | 51.74 | 5.75 | 9.00 |
| IMB-3-19 (N7 modification) | 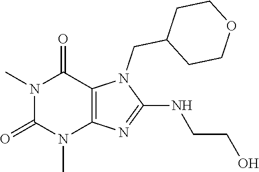 [SMILES: Cn1c(=O)c2c(nc(NCCO)n2CC2CCOCC2)n(C)c1=O] | >200 | 1.88 | >106.38 |
| IMB-4-3 (N7 modification) | 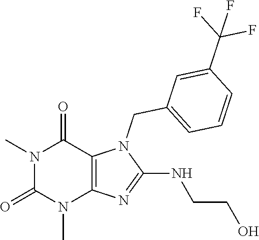 [SMILES: Cn1c(=O)c2c(nc(NCCO)n2Cc2cccc(C(F)(F)F)c2)n(C)c1=O] | >200 | 3.15 | >63.49 |
| IMB-4-6 (N7 modification) | 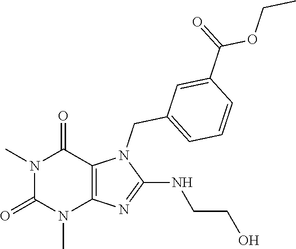 [SMILES: CCOC(=O)c1cccc(Cn2c(NCCO)nc3c2c(=O)n(C)c(=O)n3C)c1] | 200 | 2.47 | 80.97 |
| IMB-4-12 (N7 modification) | 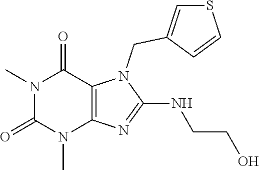 [SMILES: Cn1c(=O)c2c(nc(NCCO)n2Cc2ccsc2)n(C)c1=O] | >200 | 3.99 | >50.13 |
| IMB-4-13 (N7 modification) | 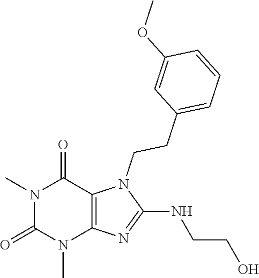 [SMILES: COc1cccc(CCn2c(NCCO)nc3c2c(=O)n(C)c(=O)n3C)c1] | >200 | 15.41 | >12.98 |
| IMB-ZHB-4x (N7 modification) | 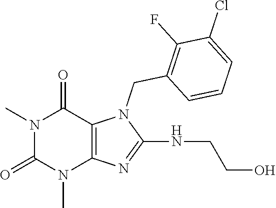 [SMILES: Cn1c(=O)c2c(nc(NCCO)n2Cc2cccc(Cl)c2F)n(C)c1=O] | 90.97 | 3.78 | 24.08 |
| IMB-ZHB-13x (N7 modification) | 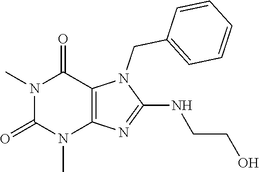 [SMILES: Cn1c(=O)c2c(nc(NCCO)n2Cc2ccccc2)n(C)c1=O] | >151.93 | 5.62 | >27.03 |
| IMB-2-2 (N7 modification) | 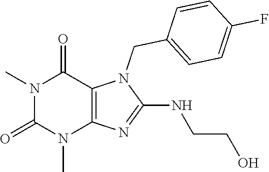 [SMILES: Cn1c(=O)c2c(nc(NCCO)n2Cc2ccc(F)cc2)n(C)c1=O] | >200 | 3.15 | >63.49 |
| IMB-2-3 (N7 modification) | 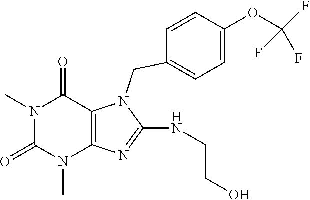 [SMILES: Cn1c(=O)c2c(nc(NCCO)n2Cc2ccc(OC(F)(F)F)cc2)n(C)c1=O] | >241.90 | 11.44 | >21.14 |
| IMB-2-5 (N7 modification) | 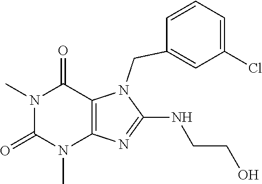 [SMILES: Cn1c(=O)c2c(nc(NCCO)n2Cc2cccc(Cl)c2)n(C)c1=O] | 200 | 1.33 | 150.38 |
| IMB-2-6 (N7 modification) | 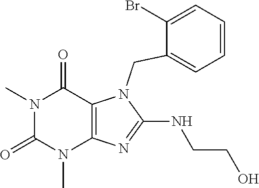 [SMILES: Cn1c(=O)c2c(nc(NCCO)n2Cc2ccccc2Br)n(C)c1=O] | >200 | 12.83 | >12.59 |
| IMB-2-8 (N7 modification) | 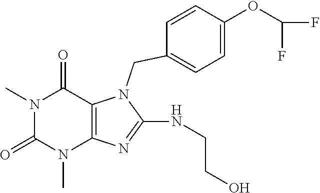 [SMILES: Cn1c(=O)c2c(nc(NCCO)n2Cc2ccc(OC(F)F)cc2)n(C)c1=O] | >200 | 2.47 | >80.97 |
| IMB-2-14 (N7 modification) | 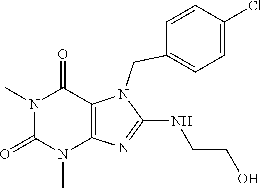 [SMILES: Cn1c(=O)c2c(nc(NCCO)n2Cc2ccc(Cl)cc2)n(C)c1=O] | 200 | 1.43 | 139.86 |
| IMB-68 (N7 modification) | 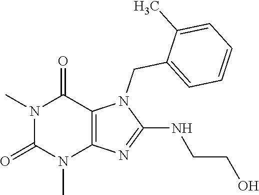 [SMILES: Cc1ccccc1Cn1c(NCCO)nc2c1c(=O)n(C)c(=O)n2C] | 200 | 1.43 | 139.86 |
| IMB-79 (N7 modification) | 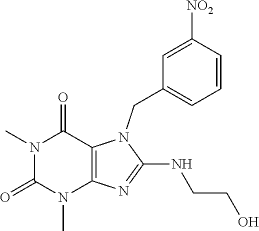 [SMILES: Cn1c(=O)c2c(nc(NCCO)n2Cc2cccc([N+](=O)[O-])c2)n(C)c1=O] | >200 | 17.24 | >11.60 |
| IMB-80 (N7 modification) | 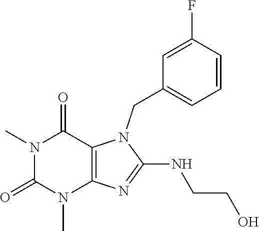 [SMILES: Cn1c(=O)c2c(nc(NCCO)n2Cc2cccc(F)c2)n(C)c1=O] | >287.85 | 10.65 | >27.03 |
| IMB-81 (N7 modification) | 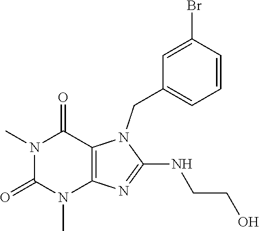 [SMILES: Cn1c(=O)c2c(nc(NCCO)n2Cc2cccc(Br)c2)n(C)c1=O] | 123.68 | 2.47 | 50.07 |
| IMB-82 (N7 modification) | 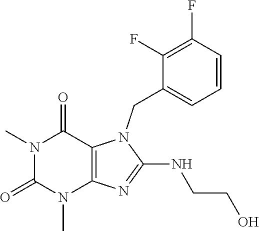 [SMILES: Cn1c(=O)c2c(nc(NCCO)n2Cc2cccc(F)c2F)n(C)c1=O] | >273.75 | 30.41 | >9 |
| IMB-83 (N7 modification) | 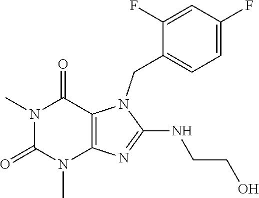 [SMILES: Cn1c(=O)c2c(nc(NCCO)n2Cc2ccc(F)cc2F)n(C)c1=O] | >200 | 3.15 | >63.49 |
| IMB-84 (N7 modification) | 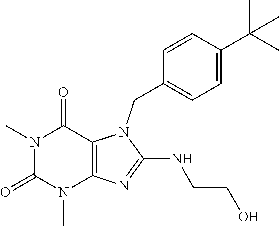 [SMILES: Cn1c(=O)c2c(nc(NCCO)n2Cc2ccc(C(C)(C)C)cc2)n(C)c1=O] | >259.4 | 15.38 | >16.86 |
| IMB-85 (N7 modification) | 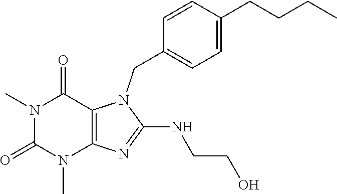 [SMILES: CCCCc1ccc(Cn2c(NCCO)nc3c2c(=O)n(C)c(=O)n3C)cc1] | 81.41 | 0.09 | 904.56 |
| IMB-88 (N7 modification) | 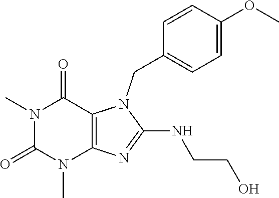 | >278.24 | 16.50 | >16.86 |
| IMB-92 (N7 modification) | 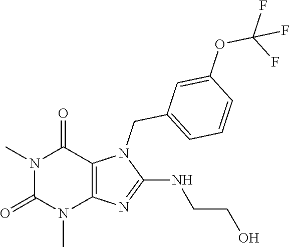 [SMILES: Cn1c(=O)c2c(nc(NCCO)n2Cc2cccc(OC(F)(F)F)c2)n(C)c1=O] | 143.84 | 0.64 | 224.75 |
| IMB-93 (N7 modification) | 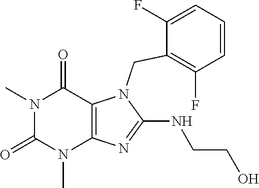 [SMILES: Cn1c(=O)c2c(nc(NCCO)n2Cc2c(F)cccc2F)n(C)c1=O] | >200 | 2.47 | >80.97 |
| IMB-3-6 (N1 modification) | 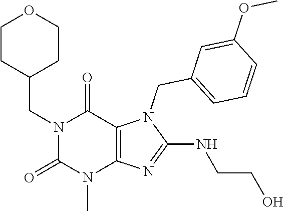 [SMILES: COc1cccc(Cn2c(NCCO)nc3c2c(=O)n(CC2CCOCC2)c(=O)n3C)c1] | >200 | / | / |
| IMB-3-13 (N1 modification) | 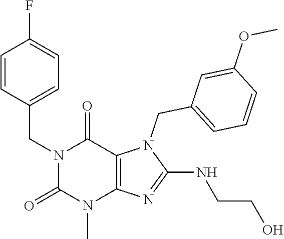 [SMILES: COc1cccc(Cn2c(NCCO)nc3c2c(=O)n(Cc2ccc(F)cc2)c(=O)n3C)c1] | 46.22 | 17.24 | 2.68 |
| IMB-3-15 (N1 modification) | 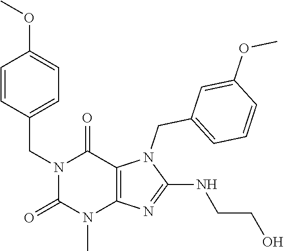 [SMILES: COc1ccc(Cn2c(=O)c3c(nc(NCCO)n3Cc3cccc(OC)c3)n(C)c2=O)cc1] | >200 | 17.24 | >11.60 |
| IMB-3-16 (N1 modification) | 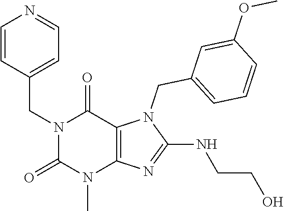 [SMILES: COc1cccc(Cn2c(NCCO)nc3c2c(=O)n(Cc2ccncc2)c(=O)n3C)c1] | 115.47 | 22.22 | >5.20 |
| IMB-3-30 (N1 modification) | 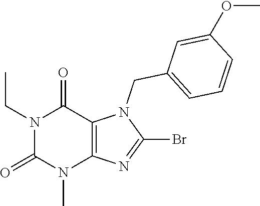 [SMILES: CCn1c(=O)c2c(nc(Br)n2Cc2cccc(OC)c2)n(C)c1=O] | 155.21 | 4.28 | 36.26 |
| IMB-3-45 (N1 modification) | 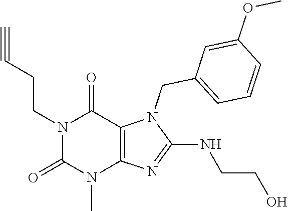 [SMILES: C#CCCn1c(=O)c2c(nc(NCCO)n2Cc2cccc(OC)c2)n(C)c1=O] | >200 | 66.67 | >3 |
| IMB-3-46 (N1 modification) | 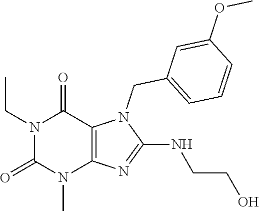 [SMILES: CCn1c(=O)c2c(nc(NCCO)n2Cc2cccc(OC)c2)n(C)c1=O] | >200 | 155.21 | >1.29 |
| IMB-3-47 (N1 modification) | 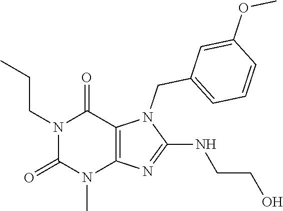 [SMILES: CCCn1c(=O)c2c(nc(NCCO)n2Cc2cccc(OC)c2)n(C)c1=O] | >200 | 85.90 | >2.33 |
| IMB-3-57 (N1 modification) | 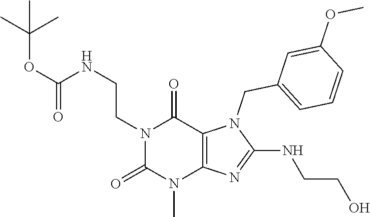 [SMILES: COc1cccc(Cn2c(NCCO)nc3c2c(=O)n(CCNC(=O)OC(C)(C)C)c(=O)n3C)c1] | >200 | 66.67 | >3.00 |
| IMB-3-58 (N1 modification) | 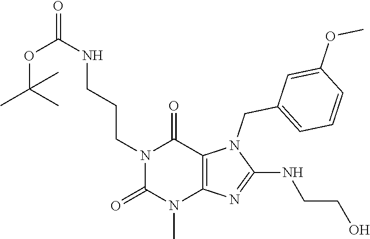 [SMILES: COc1cccc(Cn2c(NCCO)nc3c2c(=O)n(CCCNC(=O)OC(C)(C)C)c(=O)n3C)c1] | >200 | 66.67 | >3.00 |
| IMB-3-71 (N1 modification) | 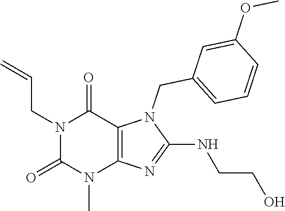 [SMILES: C=CCn1c(=O)c2c(nc(NCCO)n2Cc2cccc(OC)c2)n(C)c1=O] | 115.47 | 38.49 | 3.00 |
| IMB-3-72 (N1 modification) | 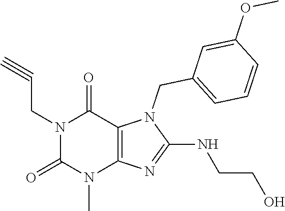 [SMILES: C#CCn1c(=O)c2c(nc(NCCO)n2Cc2cccc(OC)c2)n(C)c1=O] | >200 | 115.47 | >1.73 |
| IMB-3-81 (N3 modification) | 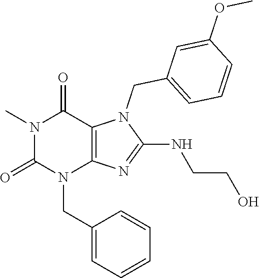 [SMILES: COc1cccc(Cn2c(NCCO)nc3c2c(=O)n(C)c(=O)n3Cc2ccccc2)c1] | >200 | 96.15 | >2.08 |
| RBV | 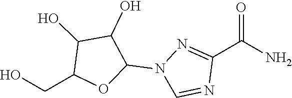 | 317.8 | 26.2 | 12.1 |
| Molnupiravir | 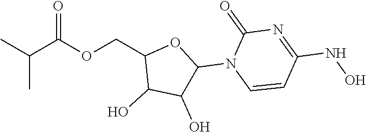 [SMILES: CC(C)C(=O)OCC1OC(n2ccc(NO)nc2=O)C(O)C1O] | >200 | 1.92 | >104.17 |

TABLE 2
The anti-HCoV-229E activity of IMB-C5 series compounds in Huh7.5
cells determined by CPE assay

| | | HCoV-229E | | |
|---|---|---|---|---|
| Compound | Compound | TC₅₀ | EC₅₀ | |
| information | Structure | (μM) | (μM) | SI |
| IMB-C5 | 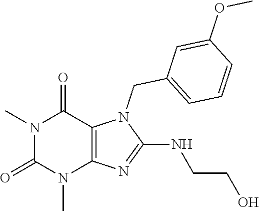 | >139.12 | 26.77 | >5.2 |
| IMB-ZH-2 | 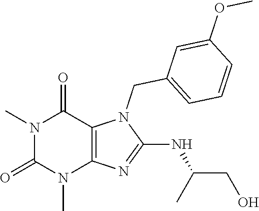 | >133.98 | 82.85 | >1.62 |
| IMB-ZH-6 | 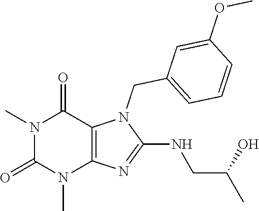 | >133.98 | 82.85 | >1.62 |
| IMB-ZH-10 | 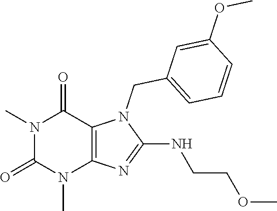 | >133.98 | 19.00 | >7.05 |
| IMB-ZH-11 | 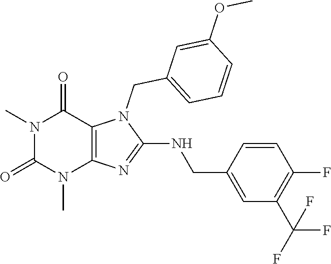 [SMILES: COc1cccc(Cn2c(NCc3ccc(F)c(C(F)(F)F)c3)nc3c2c(=O)n(C)c(=O)n3C)c1] | >101.79 | 26.59 | >3.83 |
| IMB-ZH-12 | 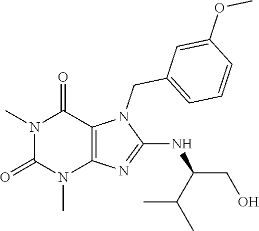 | >124.63 | 59.92 | >2.08 |
| IMB-ZHB-4x | 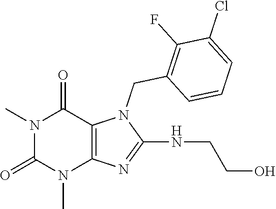 [SMILES: Cn1c(=O)c2c(nc(NCCO)n2Cc2cccc(Cl)c2F)n(C)c1=O] | 90.97 | 8.42 | 10.80 |
| IMB-ZHB-13x | 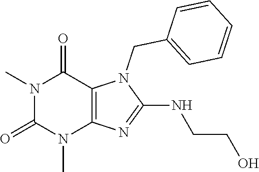 [SMILES: Cn1c(=O)c2c(nc(NCCO)n2Cc2ccccc2)n(C)c1=O] | >151.93 | 38.47 | >3.95 |
| IMB-ZHC-2 | 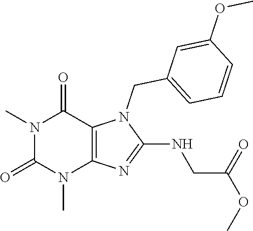 | >129.17 | 43.06 | >3 |
| IMB-ZHC-6 | 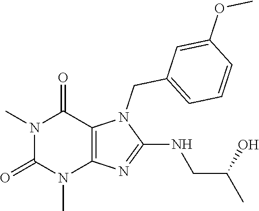 | >133.98 | 58.76 | >2.28 |
| IMB-ZHC-7 | 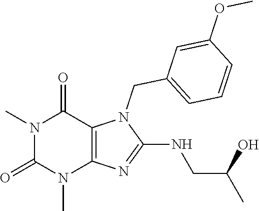 | >133.98 | 44.67 | >3 |
| IMB-ZHC-15 | 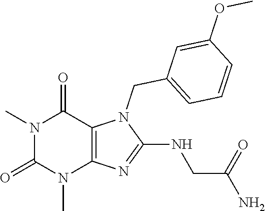 | >134.34 | 58.92 | >2.28 |
| IMB-2-3 | 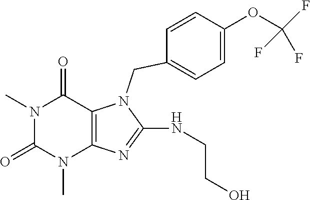 [SMILES: Cn1c(=O)c2c(nc(NCCO)n2Cc2ccc(OC(F)(F)F)cc2)n(C)c1=O] | >241.90 | 4.79 | >50.5 |
| IMB-80 | 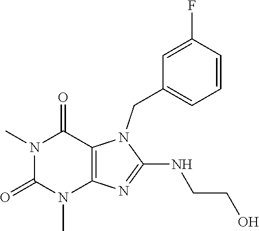 [SMILES: Cn1c(=O)c2c(nc(NCCO)n2Cc2cccc(F)c2)n(C)c1=O] | >287.85 | 14.02 | >20.53 |
| IMB-82 | 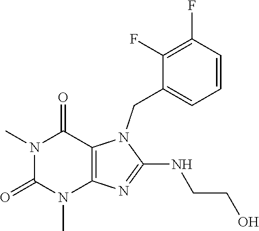 [SMILES: Cn1c(=O)c2c(nc(NCCO)n2Cc2cccc(F)c2F)n(C)c1=O] | >273.75 | 69.34 | >3.95 |
| IMB-84 | 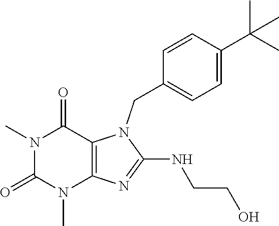 [SMILES: Cn1c(=O)c2c(nc(NCCO)n2Cc2ccc(C(C)(C)C)cc2)n(C)c1=O] | >259.4 | 12.27 | >21.14 |
| IMB-85 | 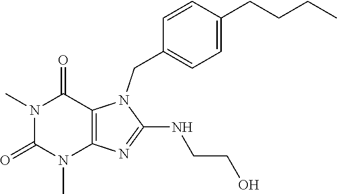 [SMILES: CCCCc1ccc(Cn2c(NCCO)nc3c2c(=O)n(C)c(=O)n3C)cc1] | >259.4 | 4.05 | >64.1 |
| IMB-88 | 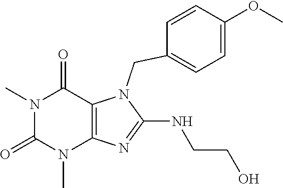 | >278.24 | 23.48 | >11.85 |
| RBV | 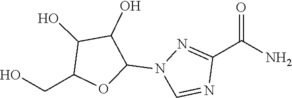 | >204.7 | 16.7 | >12.3 |
| Molnupiravir | 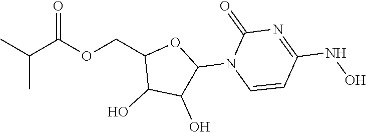 [SMILES: CC(C)C(=O)OCC1OC(n2ccc(NO)nc2=O)C(O)C1O] | >200 | 4.28 | >46.73 |

### EXAMPLE 3. DETERMINATION OF THE ANTI-α-CORONAVIRUS HCoV-229E ACTIVITY OF IMB-C5

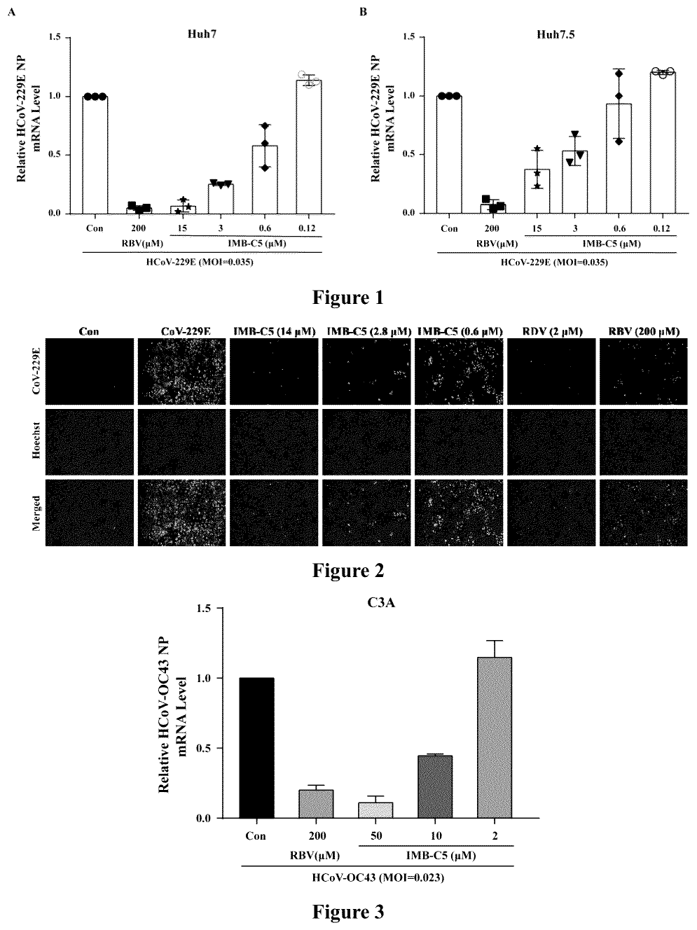

In the in vitro pharmacodynamic assay, the effect of IMB-C5 on mRNA level of coronavirus HCoV-229E N protein was first determined (FIG. 1).

Huh7 and Huh7.5 cells were infected at a viral load of MOI=0.035, and simultaneously administered with three concentrations of IMB-C5 or 200 μM of the positive drug (RBV). After 24 h, RNA was extracted for RT-qPCR. As shown in FIG. 1A and 1B, IMB-C5 decreased mRNA level of HCoV-229E N protein in both Huh7 and Huh7.5 cells in a dose-dependent manner, and exhibited a better antiviral activity in Huh7 cells than in Huh7.5 cells.

Subsequently, the effect of IMB-C5 on the double-stranded RNA (dsRNA) of HCoV-229E was determined by immunofluorescence assay (FIG. 2).

Huh7 cells were infected at a viral load of MOI=0.035, and simultaneously administered with gradient concentrations of IMB-C5 or the positive drug. After cultured at 35° C. for 24 h, the treated cells were washed with PBS for 3 times, added with 4% paraformaldehyde and incubated at room temperature for 15 min for fixation. The cells were then washed and incubated with PBS buffer containing 0.5% Triton X-100 at room temperature for 20 min for permeabilization. The cells were then washed and added with TBST buffer containing 1% bovine serum albumin (BSA) for blocking at room temperature for 1 h. The antibody against dsRNA were added and incubated at 4° C. overnight. The cells were then washed with PBST buffer, added with the FITC fluorescence-labeled secondary antibody, and incubated at room temperature in dark for 1 h. Finally, the cells were added with the nuclei-staining solution (Hoechst 33342), incubated at room temperature for 10 min, and observed under a fluorescence microscope for dsRNA level. As shown in FIG. 2, IMB-C5 had an inhibitory effect on dsRNA production during replication of HCoV-229E in Huh7 cells.

### EXAMPLE 4. DETERMINATION OF THE ANTI-β-CORONAVIRUS HCoV-OC43 ACTIVITY OF IMB-C5

C3A cells were used as viral hosts and the effect of IMB-C5 on mRNA level of coronavirus HCoV-OC43 N protein was determined (FIG. 3). CA3 cells were infected at a viral load of MOI=0.023, and simultaneously administered with three concentrations of IMB-C5 or 200 μM of the positive drug (RBV). After 24 h, RNA was extracted for RT-qPCR. The results showed that IMB-C5 decreased mRNA level of HCoV-OC43 N protein in C3A cells in a dose-dependent manner.

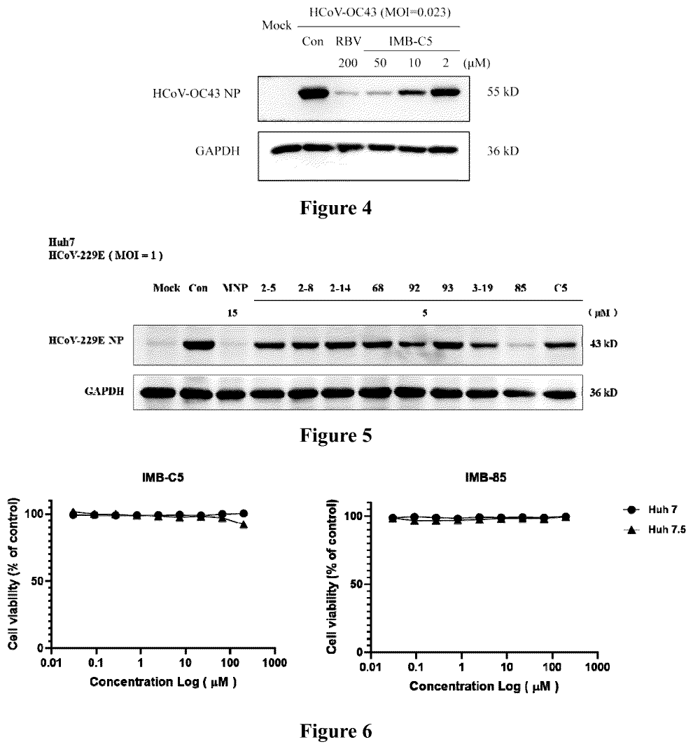

Further, the effect of IMB-C5 on N protein level of coronavirus HCoV-OC43 was determined (FIG. 4). C3A cells were infected at a viral load of MOI=0.023, and simultaneously administered with three concentrations of IMB-C5 or 200 μM of the positive drug (RBV). The viral N protein level was determined after 24 h. As shown in the figure, IMB-C5 had a good inhibitory effect on HCoV-OC43 and inhibited the expression of the viral N protein in a dose-dependent manner.

### EXAMPLE 5. INHIBITORY EFFECT OF IMB-C5 SERIES COMPOUNDS ON N PROTEIN LEVEL OF CORONAVIRUS HCoV-229E

In the in vitro pharmacodynamic assay, the effect of IMB-C5 series compounds on N protein level of coronavirus HCoV-229E was determined. 8 compounds with SI>80 were selected from the CPE results, including IMB-2-5, IMB-2-8, IMB-2-14, IMB-68, IMB-92, IMB-93, IMB-3-19 and IMB-85, and IMB-C5 was used as homologue control. Huh7 cells were infected at a viral load of MOI=1, and simultaneously administered with 5 μM of IMB-C5 series compounds and 15 μM of the positive control Molnupiravir (MNP, a commercially available oral coronavirus RdRp inhibitor). After 24 h, the protein was extracted for Western blotting. As shown in FIG. 5 (in the figure, compound IMB-2-5 is marked as 2-5, and the rest are marked in the same way), the above 8 compounds (at a concentration of 5 μM) inhibited HCoV-229E in Huh7 cells to different degrees. At the same concentration, IMB-85 had the best activity in decreasing HCoV-229E N protein level, which was significantly superior to IMB-C5 and other compounds, and was comparable to the positive control (15 μM MNP).

### EXAMPLE 6. CYTOTOXICITY ASSAY OF COMPOUNDS IMB-C5 AND IMB-85

In order to determine the possible cytotoxicity of IMB-C5 and IMB-85, the CCK-8 assay was first used to detect the effect of the two compounds at different concentrations on viability of different cells 48 hours after administration. As shown in FIG. 6, when IMB-C5 and IMB-85 were administered at a concentration of 200 μM and less, the viability of the two hepatocellular carcinoma cells (Huh7 and Huh7.5) 48 hours after administration were above 95% of the control group (TC₅₀>200 μM), indicating that both compounds have low toxicity to the above cells.

### EXAMPLE 7. INHIBITORY ACTIVITY OF IMB-C5 AND IMB-85 AGAINST CORONAVIRUS HCOV-229E

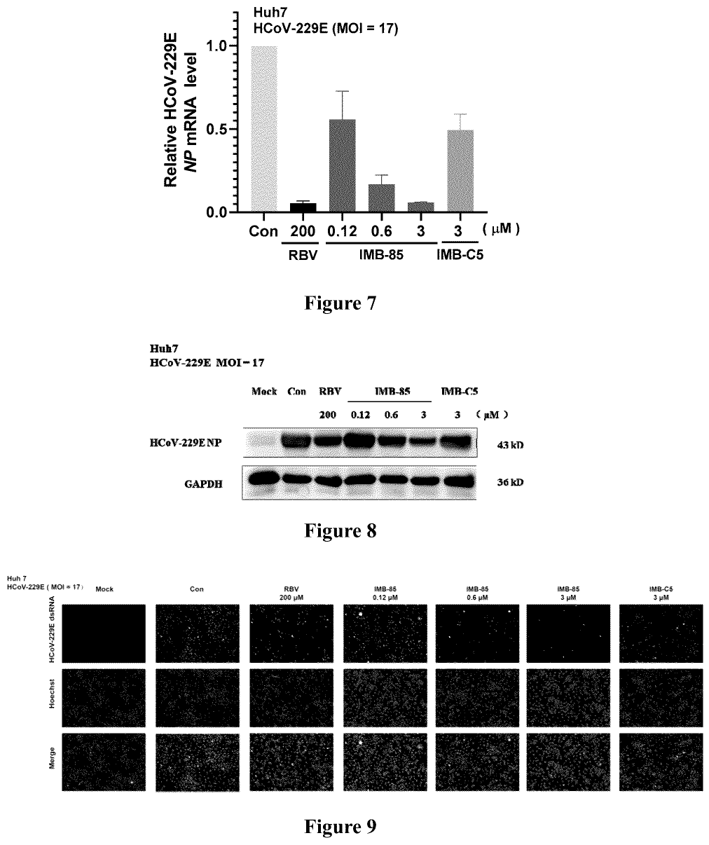

The effect of IMB-C5 and IMB-85 on mRNA level and protein level of coronavirus HCoV-229E N protein was determined. Huh7 cells were infected at a viral load of MOI=17 and simultaneously administered with IMB-C5 (3 μM), IMB-85 (3 μM, 0.6 μM and 0.12 μM) and the positive drug (RBV, 200 μM). After 24 h, RNA and protein were extracted for RT-qPCR and Western blotting, respectively. As shown in FIG. 7 and FIG. 8, IMB-85 decreased mRNA and protein level of HCoV-229E N protein in Huh7 cells in a dose-dependent manner, and exhibited significantly better activity than IMB-85 at the same concentration.

Under the same experimental conditions, the effect of IMB-C5 and IMB-85 on HCoV-229E double-stranded RNA (dsRNA) was determined by immunofluorescence assay (FIG. 9). The results showed that both IMB-C5 and IMB-85 inhibited dsRNA production during replication of HCOV-229E in Huh7 cells, and IMB-85 exhibited an activity superior to that of IMB-C5 at the same concentration.

### EXAMPLE 8. EFFECT OF IMB-C5 AND IMB-85 ON N PROTEIN LEVEL OF CORONAVIRUS HCoV-229E IN HUH7 AND HUH7.5 CELLS

### (1) Effect of IMB-C5 and IMB-85 on N Protein Level of Coronavirus HCoV-229E in Huh7 Cells

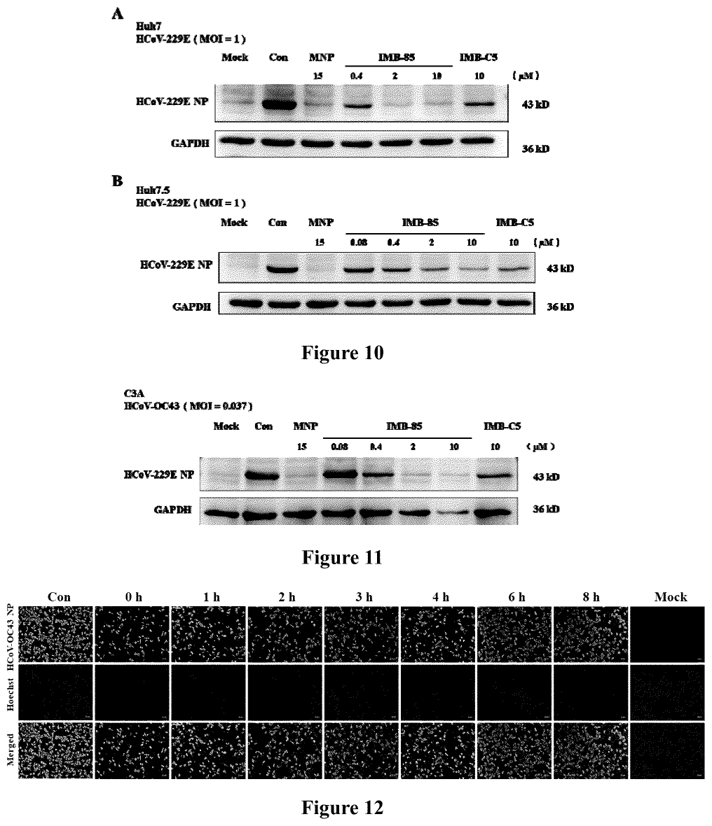

Huh7 cells were used as viral hosts and the effect of IMB-C5 and IMB-85 on N protein level of coronavirus HCoV-229E was determined (FIG. 10A). Huh7 cells were infected at a viral load of MOI=1, and simultaneously administered with different concentrations of IMB-85 and IMB-C5, with MNP as a positive control. After 24 h, total protein in the cells was extracted for Western blotting. The results showed that IMB-85 decreased the HCoV-229E N protein level in Huh7 cells in a dose-dependent manner, and exhibited a better inhibitory effect on coronavirus than IMB-C5 at the same concentration (10 μM) and a comparable effect to the positive control (MNP, 15 M).

### (2) Effect of IMB-C5 and IMB-85 on N Protein Level of Coronavirus HCoV-229E in Huh7.5 Cells

Huh7.5 cells were used as viral hosts and the effect of IMB-C5 and IMB-85 on N protein level of coronavirus HCoV-229E was determined (FIG. 10B). Huh7.5 cells were infected at a viral load of MOI=1, and simultaneously administered with different concentrations of IMB-85 and IMB-C5, with MNP as the positive control. After 24 h, total protein in the cells was extracted for Western blotting. The results showed that IMB-85 also decreased the HCoV-229E N protein level in Huh7.5 cells in a dose-dependent manner, and exhibited a better inhibitory effect on coronavirus than IMB-C5 and a comparable effect to that of the positive control (MNP, 15 μM).

### EXAMPLE 9. INHIBITORY EFFECT OF IMB-C5 AND IMB-85 ON N PROTEIN LEVEL OF Coronavirus HCoV-OC43

The effect of IMB-C5 and IMB-85 on N protein level of coronavirus HCoV-OC43 was determined. C3A cells were infected at a viral load of MOI=0.037, and simultaneously administered with IMB-C5 (10 μM), IMB-85 (10 μM, 2 μ, 0.4 μM and 0.08 μM) and MNP (the positive control, 15 μM). After 24 h, the protein was extracted for Western blotting. As shown in FIG. 11, IMB-85 decreased the HCoV-OC43 N protein level in C3A cells in a dose-dependent manner.

### EXAMPLE 10. DETERMINATION OF THE ANTI-SARS-COV-2 ACTIVITY OF IMB-C5

The anti-SARS-COV-2 activity of IMB-C5 was determined by CPE assay. Vero E6 cells were seeded into 96-well culture plates and cultured at 37° C. overnight. The culture medium was then discarded. The cells in the 96-well culture plates were infected with SARS-COV-2 Beta variant (MOI=0.05) and simultaneously administered with the compounds diluted in the DMEM medium without FBS. After 1 h of infection, the culture medium was discarded, and the cells in the 96-well culture plates were administered with the compounds diluted in DMEM medium containing 2% FBS at a concentration gradient and cultured. Cytopathic effect in each group was observed when CPE in the virus control group reached 4+. The results showed that IMB-C5 had an inhibitory activity against the SARS-COV-2 Beta variant.

TABLE 3
The anti-SARS-CoV-2 activity of IMB-C5 in
Vero E6 cells determined by CPE assay

| | TC₅₀ | IC₅₀ | |
|---|---|---|---|
| Compound | (μM) | (μM) | SI |
| IMB-C5 | 493.3 | 34.8 | 14.2 |
| Remdesivir | >200 | 0.73 | >274.0 |

### EXAMPLE 11. IMB-C5 AND IMB-85 FUNCTIONING AT THE EARLY STAGE OF CORONAVIRUS INFECTION

C3A cells were infected with HCoV-OC43 (MOI=0.28) and then added with 50 μM IMB-C5 at different time points. The viral N protein level was determined by immunofluorescence assay (FIG. 12). The results showed that IMB-C5 exhibited the most significant inhibitory effect on virus when it was administered at the time of virus infection, exhibited a better antiviral effect when it was administered 1 h to 4 h after infection, and exhibited a greatly diminished antiviral effect when it was administered 6 h or more after infection. The results suggested that IMB-C5 might function at the early stage of coronavirus infection.

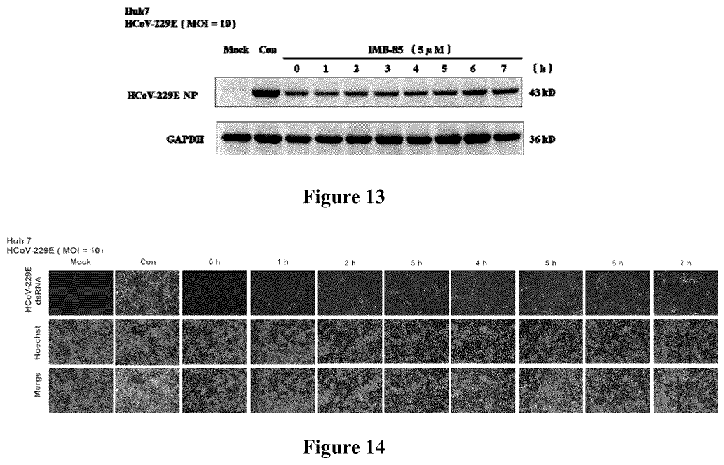

Huh7 cells were infected with HCoV-229E (MOI=10) for 2 h, and added with 5 M IMB-85 at the time of infection and at different time points after infection. The viral N protein level (FIG. 13) and dsRNA level (FIG. 14) were determined by Western blotting and immunofluorescence assay, respectively. The results showed that IMB-85 exhibited a significant inhibitory effect on virus when it was administered at the time of infection or within 1-5 h after infection, and exhibited a diminished antiviral effect when it was administered 6 h or more after infection. The results suggested that IMB-85 might function mainly at the early stage of coronavirus infection.

Finally, it should be noted that the above examples are provided only to assist those skilled in the art in understanding the essence of the present disclosure, but not to limit the protection scope of the present disclosure.

1. A compound with coronavirus inhibitory activity represented by Formula (1), 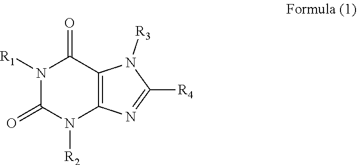
wherein,
R₁ is C1-C5 alkyl; C1-C5 monoalkenyl; C1-C5 monoalkynyl; alkyl comprising pyridine ring(s), benzene ring(s) or substituted benzene ring(s); or Boc-NH-linked C1-C3 alkyl;
R₂ is methyl or benzyl;
R₃ is epoxy hexane methyl; formate benzyl; methoxyl; fluoro-methoxyl or fluoro-methyl modified benzyl; monohalogenated or polyhalogenated benzyl; C1-C5 alkyl substituted benzyl; nitro-substituted benzyl; thenyl; or substituted phenylethyl;
R₄ is —NH—R₅; benzyl or substituted benzyl; imidazolyl; or substituted piperazinyl;
R₅ is straight or branched C1-C5 hydroxyalkyl, alkoxy alkyl; halogen atom and/or methyl or fluoro-methyl substituted benzene ring and/or benzyl; amide-containing alkyl; or ester-containing alkyl.

2. The compound of claim 1, wherein the compound is IMB-ZH-2, IMB-ZH-11, IMB-ZH-12, IMB-ZHC-2, IMB-ZHC-15, IMG-2-26, IMB-2-31, IMB-2-32, IMB-3-19, IMB-4-6, IMB-4-12, IMB-4-13, IMB-ZHB-4x, IMB-2-3, IMB-2-8, IMB-68, IMB-82, IMB-83, IMB-84, IMB-85, IMB-92, IMB-93, IMB-3-6, IMB-3-13, IMB-3-15, IMB-3-16, IMB-3-30, IMB-3-45, IMB-3-46, IMB-3-47, IMB-3-57, IMB-3-58, IMB-3-71, IMB-3-72 or IMB-3-81.

3. (canceled)

4. A pharmaceutical composition and/or formulation comprising a therapeutically effective amount of the compound of claim 1 and an optional pharmaceutical excipient or diluent.

5. A method for treating a disease caused by coronavirus infection, the method comprising administering to a patient a therapeutically effective amount of the compound of claim 1 or a pharmaceutical composition and/or formulation comprising the compound of claim 1.

6. A method for synthesizing the compound of claim 1, the method comprising three synthetic routes as follows: 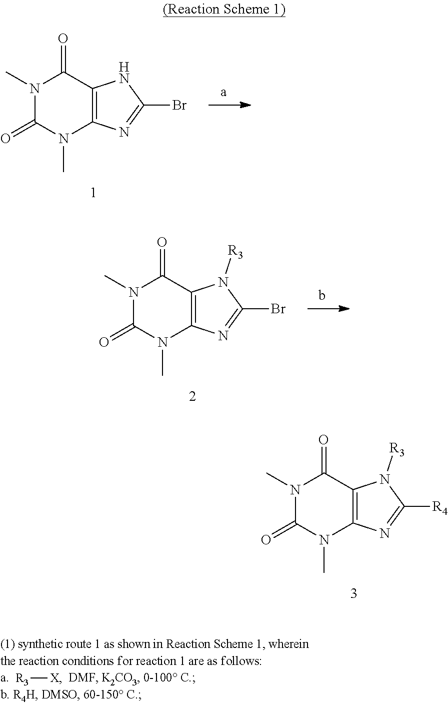
or (2) synthetic route 2 as shown in Reaction Scheme 2: 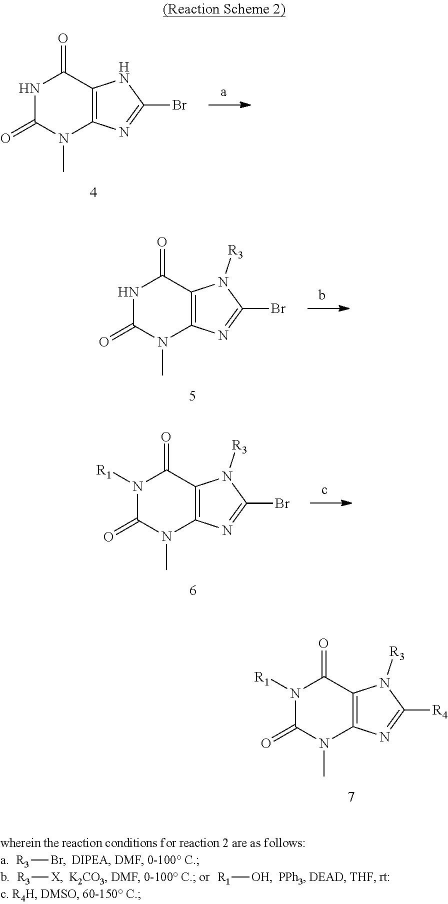
or (3) synthetic route 3 as shown in Reaction Scheme 3:

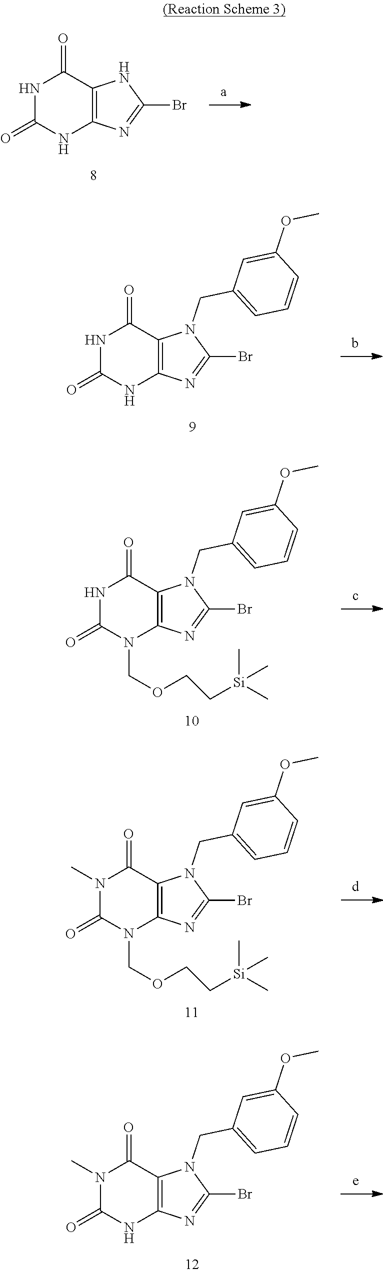 [SMILES: O=c1[nH]c(=O)c2[nH]c(Br)nc2[nH]1 ; COc1cccc(Cn2c(Br)nc3[nH]c(=O)[nH]c(=O)c32)c1 ; COc1cccc(Cn2c(Br)nc3c2c(=O)[nH]c(=O)n3COCC[Si](C)(C)C)c1 ; COc1cccc(Cn2c(Br)nc3c2c(=O)n(C)c(=O)n3COCC[Si](C)(C)C)c1 ; COc1cccc(Cn2c(Br)nc3[nH]c(=O)n(C)c(=O)c32)c1]

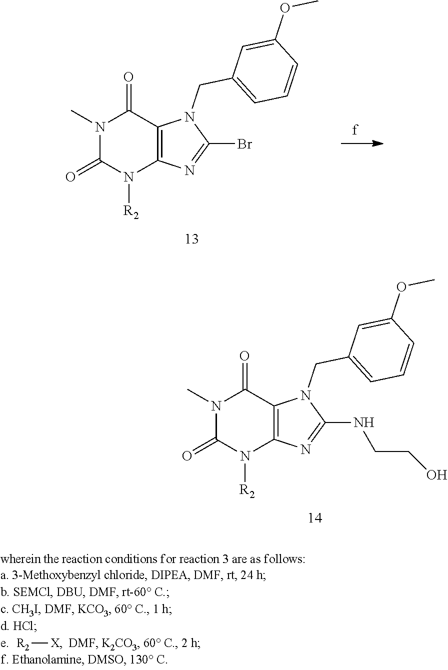

7. The method of claim 5, wherein the coronavirus is HCOV-229E, HCoV-OC43 or SARS-COV-2.
